SEQUENCE LISTING (18 sequences)

SEQ ID NO: 1 - DNA, 1782 bp, Homo sapiens
  source 1..1782: mol_type = other DNA
atggccgctgcgtcctcatcggattccgacgcctgcggagctgagagcaatgaggccaattcgaagtggttggatgcgcactacgacccaatggccaatatccacaccttttctgcctgcctagcgctggcagatttacatggggatggggaatacaagctggtggtaggggaccttggccctggtgggcagcagccccgcctgaaggtgctcaaaggaccactggtgatgaccgaaagcccgctacctgctctgccagctgctgctgccaccttcctcatggagcaacatgagccccggaccccagctctggcacttgcttcaggcccttgtgtctatgtgtataagaatctcagaccctacttcaagttcagcctgccccaattgcctccaaatcctctggaacaagacctttggaaccaggccaaagaggaccgaatcgaccccttaaccctgaaggagatgctggagagcatccgggagacggcagaggagcctttgtccatccagtcactcaggtttctgcagctggagctaagtgaaatggaggcatttgtaaaccaacacaagtccaactccatcaagcggcagacagtcatcaccaccatgaccaccttgaagaagaacctggctgacgaggatgctgtgtcttgcctggtgctgggcaccgagaacaaggagctcctggtgcttgaccccgaggccttcaccattttagccaagatgagccttcccagcgtccccgtcttcctagaggtttctggccagtttgatgttgagttccggcttgccgcggcctgccgcaatggaaacatctatattctgagaagagactccaagcaccccaagtactgcatcgagctgagcgcccagcctgtgggacttatccgggtacacaaggtcctagtggtgggcagcacccaagacagcctgcatggcttcacccacaaggggaagaagctgtggacagtgcagatgcccgcagccatcctgaccatgaacctcctggagcagcattcccggggcctgcaggccgtcatggctgggctggccaatggagaggtccgcatttatcgtgacaaggccctgctcaatgtcatccacaccccggatgcagtgaccagcctttgctttggccggtacgggcgggaggacaacaccctcatcatgaccactcgaggtggtggcctgatcatcaagatcctgaagcgtacagcagtgtttgtagagggaggaagtgaggtgggtcccccaccagcccaggccatgaaactcaatgtgccccgaaagacccggctttacgtggatcagacactgcgagagcgggaggctggcaccgccatgcaccgggccttccagacagacctatacctgctgcgcctacgtgctgcccgcgcctacctgcaggccctcgagtccagcctgagccccctgtccacgacagcccgagagccactcaagctgcacgccgtggttcagggccttggccccacctttaagctcacacttcacctgcagaacacctcaacaacccgtcctgtcctggggctgctggtctgcttcctgtacaacgaggcgctctattccctgccccgggccttcttcaaggtacccttgctggtgccagggctcaactaccccctggagacctttgtggagagtctcagtaacaagggcatctcagacatcatcaaggtgctggtgcttcgagaaggccaaagtgcacccctgctgagtgcccacgtcaacatgcctgggagcgaggggctggcggccgcctga

SEQ ID NO: 2 - protein, 593 aa, Homo sapiens
MAAASSSDSDACGAESNEANSKWLDAHYDPMANIHTFSACLALADLHGDGEYKLVVGDLGPGGQQPRLKVLKGPLVMTESPLPALPAAAATFLMEQHEPRTPALALASGPCVYVYKNLRPYFKFSLPQLPPNPLEQDLWNQAKEDRIDPLTLKEMLESIRETAEEPLSIQSLRFLQLELSEMEAFVNQHKSNSIKRQTVITTMTTLKKNLADEDAVSCLVLGTENKELLVLDPEAFTILAKMSLPSVPVFLEVSGQFDVEFRLAAACRNGNIYILRRDSKHPKYCIELSAQPVGLIRVHKVLVVGSTQDSLHGFTHKGKKLWTVQMPAAILTMNLLEQHSRGLQAVMAGLANGEVRIYRDKALLNVIHTPDAVTSLCFGRYGREDNTLIMTTRGGGLIIKILKRTAVFVEGGSEVGPPPAQAMKLNVPRKTRLYVDQTLREREAGTAMHRAFQTDLYLLRLRAARAYLQALESSLSPLSTTAREPLKLHAVVQGLGPTFKLTLHLQNTSTTRPVLGLLVCFLYNEALYSLPRAFFKVPLLVPGLNYPLETFVESLSNKGISDIIKVLVLREGQSAPLLSAHVNMPGSEGLAAA

SEQ ID NO: 3 - DNA, 584 bp, synthetic construct
  misc_feature 1..584: note = CAG promoter sequence
  source 1..584: mol_type = other DNA
gcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 4 - DNA, 528 bp, synthetic construct
  misc_feature 1..528: note = CAG promoter sequence
  source 1..528: mol_type = other DNA
ccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 5 - DNA, 511 bp, Human cytomegalovirus
  source 1..511: mol_type = other DNA
gatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggtcattattgacgtcaatgggcgggggtcgttgggcggtcagccaggcgggccatttaccgtaagttatgtaacggg

SEQ ID NO: 6 - DNA, 204 bp, Human cytomegalovirus
  source 1..204: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 7 - DNA, 240 bp, Homo sapiens
  source 1..240: mol_type = other DNA
gggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccgggctcccaggggcttcccagtggtccccagggaaccctcgaca

SEQ ID NO: 8 - DNA, 1782 bp, synthetic construct
  misc_feature 1..1782: note = Codon optimised nucleotide sequence encoding human BBS1 protein
  source 1..1782: mol_type = other DNA
atggctgccgccagcagttctgattctgatgcctgtggcgccgagagcaacgaggccaattctaaatggctggacgcccactacgaccccatggccaatatccacacctttagcgcctgtctggccctggctgatcttcatggcgacggcgagtataagctggttgtgggagatcttggacctggcggacagcagcctagactgaaggtgctgaagggccctctcgtgatgacagagtctcctcttcctgctctgcctgccgccgctgccacatttctgatggaacagcacgagcccagaacacccgctctggctcttgcttctggcccttgcgtgtacgtgtacaagaacctgcggccttacttcaagttcagcctgcctcagctgcctcctaatcctctggaacaggacctgtggaaccaggccaaagaggacagaatcgaccctctgacactgaaagagatgctggaatccatcagagagacagccgaggaacccctgtctatccagagcctgagattcctgcagctggaactgagcgagatggaagccttcgtgaaccagcacaagagcaacagcatcaagcggcagaccgtgatcaccaccatgaccacactgaagaagaacctggccgacgaggatgccgtgtcttgtctggtgctgggcaccgagaacaaagagctgctggttctggatcccgaggccttcacaatcctggccaagatgtctctgcctagcgtgcccgtgtttctggaagtgtccggccagttcgacgtggaatttcggctggccgctgcctgcagaaacggcaacatctacatcctgcggagggacagcaagcaccccaagtactgtatcgagctgtctgcccagcctgtgggcctgattagagtgcacaaggtgctggtcgtgggcagcacacaggatagcctgcacggctttacccacaagggcaagaaactgtggaccgtgcagatgccagccgccatcctgaccatgaatctgctcgaacagcacagcagaggactgcaggctgttatggcaggactggctaatggcgaagtgcggatctacagagacaaggccctgctgaacgtgatccacacacctgatgccgtgacaagcctgtgcttcggcagatacggcagagaggacaacaccctgatcatgacaacaagaggcggcggactgatcatcaagatcctgaagagaaccgccgtgttcgtggaaggcggatctgaagttggacctcctccagctcaggccatgaagctgaatgtgcccagaaagacccggctgtacgtggaccagacactgagagaaagagaagccggcacagccatgcacagagccttccagactgacctgtacctgctgagactgagagccgccagagcctatctgcaggccctggaatctagcctgtctcctctgagcacaaccgccagagagcctctgaaactgcacgctgtggttcaaggcctgggacctaccttcaagctgaccctgcatctgcagaacaccagcaccacaagaccagtgctgggcctgctcgtgtgcttcctgtacaatgaggccctgtacagcctgccacgggccttttttaaggtgccactgctggtgcccggcctgaactaccctctggaaacctttgtggaaagcctgagcaacaagggcatcagcgacatcatcaaagtgctggtgctgagagagggccagtctgctcctctgctcagcgcccatgtgaatatgcctggctctgaaggcctggcagccgcttaa

SEQ ID NO: 9 - DNA, 1782 bp, synthetic construct
  misc_feature 1..1782: note = Codon optimised nucleotide sequence encoding human BBS1 protein
  source 1..1782: mol_type = other DNA
atggctgccgcctctagctctgactctgatgcatgtggagcagagtctaacgaggccaatagcaagtggctggacgcccactacgatcctatggccaacatccacacattctctgcctgcctggccctggcagacctgcacggcgatggagagtataagctggtggtgggcgacctgggacctggcggccagcagccacggctgaaggtgctgaagggccctctggtcatgacagagtccccactgcccgccctgccagccgccgccgccaccttcctgatggagcagcacgagcctagaaccccagccctggccctggcctctggcccctgcgtgtacgtgtataagaacctgcggccctacttcaagtttagcctgccacagctgccccctaaccctctggagcaggatctgtggaatcaggccaaggaggacaggatcgatcctctgacactgaaggagatgctggagagcatccgggagacagccgaggagccactgagcatccagtccctgagattcctgcagctggagctgtccgagatggaggcctttgtgaaccagcacaagtctaatagcatcaagcgccagaccgtgatcaccacaatgaccacactgaagaagaacctggccgacgaggatgccgtgtcttgtctggtgctgggcacagagaataaggagctgctggtgctggacccagaggccttcaccatcctggccaagatgtctctgccctctgtgcccgtgttcctggaggtgagcggacagttcgacgtggagtttcggctggctgccgcctgcagaaacggcaatatctacatcctgcggagagatagcaagcacccaaagtattgtattgagctgtccgcccagcctgtgggcctgatcagagtgcacaaggtgctggtggtgggcagcacccaggactccctgcacggcttcacacacaagggcaagaagctgtggaccgtgcagatgcccgccgccatcctgaccatgaacctgctggagcagcacagcagaggcctgcaggccgtgatggcaggcctggcaaatggagaggtgaggatctaccgcgacaaggccctgctgaatgtgatccacacccctgatgccgtgacatccctgtgcttcggccggtatggcagagaggataacacactgatcatgaccacacggggcggcggcctgatcatcaagatcctgaagagaacagccgtgtttgtggagggcggctctgaagtgggcccaccccctgcccaggccatgaagctgaatgtgcccaggaagacccgcctgtacgtggaccagacactgagggaaagagaggcaggaacagcaatgcacagggccttccagaccgatctgtacctgctgagactgagagcagcaagagcctatctgcaggccctggagagctccctgtccccactgtctaccacagcaagagaacccctgaagctgcacgcagtggtgcagggcctgggacccaccttcaagctgacactgcacctgcagaacacatccaccacaaggcctgtgctgggcctgctggtgtgcttcctgtacaatgaggccctgtattctctgccacgcgccttctttaaggtgccactgctggtgcccggcctgaactatcccctggagacattcgtggagtccctgtctaataagggcatctctgatatcatcaaggtgctggtgctgagagaaggacagtccgcccctctgctgtctgcccacgtgaatatgccaggcagcgagggcctggctgccgcctga

SEQ ID NO: 10 - DNA, 2320 bp, synthetic construct
  misc_feature 1..2320: note = Construct comprising CMV promoter and wild type BBS1 nucleotidesequence
  source 1..2320: mol_type = other DNA
ggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatccgctagcgctaccggactcagatctcgagctcaagcttcgaattccgatggccgctgcgtcctcatcggattccgacgcctgcggagctgagagcaatgaggccaattcgaagtggttggatgcgcactacgacccaatggccaatatccacaccttttctgcctgcctagcgctggcagatttacatggggatggggaatacaagctggtggtaggggaccttggccctggtgggcagcagccccgcctgaaggtgctcaaaggaccactggtgatgaccgaaagcccgctacctgctctgccagctgctgctgccaccttcctcatggagcaacatgagccccggaccccagctctggcacttgcttcaggcccttgtgtctatgtgtataagaatctcagaccctacttcaagttcagcctgccccaattgcctccaaatcctctggaacaagacctttggaaccaggccaaagaggaccgaatcgaccccttaaccctgaaggagatgctggagagcatccgggagacggcagaggagcctttgtccatccagtcactcaggtttctgcagctggagctaagtgaaatggaggcatttgtaaaccaacacaagtccaactccatcaagcggcagacagtcatcaccaccatgaccaccttgaagaagaacctggctgacgaggatgctgtgtcttgcctggtgctgggcaccgagaacaaggagctcctggtgcttgaccccgaggccttcaccattttagccaagatgagccttcccagcgtccccgtcttcctagaggtttctggccagtttgatgttgagttccggcttgccgcggcctgccgcaatggaaacatctatattctgagaagagactccaagcaccccaagtactgcatcgagctgagcgcccagcctgtgggacttatccgggtacacaaggtcctagtggtgggcagcacccaagacagcctgcatggcttcacccacaaggggaagaagctgtggacagtgcagatgcccgcagccatcctgaccatgaacctcctggagcagcattcccggggcctgcaggccgtcatggctgggctggccaatggagaggtccgcatttatcgtgacaaggccctgctcaatgtcatccacaccccggatgcagtgaccagcctttgctttggccggtacgggcgggaggacaacaccctcatcatgaccactcgaggtggtggcctgatcatcaagatcctgaagcgtacagcagtgtttgtagagggaggaagtgaggtgggtcccccaccagcccaggccatgaaactcaatgtgccccgaaagacccggctttacgtggatcagacactgcgagagcgggaggctggcaccgccatgcaccgggccttccagacagacctatacctgctgcgcctacgtgctgcccgcgcctacctgcaggccctcgagtccagcctgagccccctgtccacgacagcccgagagccactcaagctgcacgccgtggttcagggccttggccccacctttaagctcacacttcacctgcagaacacctcaacaacccgtcctgtcctggggctgctggtctgcttcctgtacaacgaggcgctctattccctgccccgggccttcttcaaggtacccttgctggtgccagggctcaactaccccctggagacctttgtggagagtctcagtaacaagggcatctcagacatcatcaaggtgctggtgcttcgagaaggccaaagtgcaccctgctgagtgcccacgtcaacatgcctgggagcgaggggctggcggccgcctgagactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtcc

SEQ ID NO: 11 - DNA, 2422 bp, synthetic construct
  misc_feature 1..2422: note = Construct comprising CMV promoter and COSEQ1-BBS1 nucleotidesequence
  source 1..2422: mol_type = other DNA
actagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgaattcgccaccatggctgccgccagcagttctgattctgatgcctgtggcgccgagagcaacgaggccaattctaaatggctggacgcccactacgaccccatggccaatatccacacctttagcgcctgtctggccctggctgatcttcatggcgacggcgagtataagctggttgtgggagatcttggacctggcggacagcagcctagactgaaggtgctgaagggccctctcgtgatgacagagtctcctcttcctgctctgcctgccgccgctgccacatttctgatggaacagcacgagcccagaacacccgctctggctcttgcttctggcccttgcgtgtacgtgtacaagaacctgcggccttacttcaagttcagcctgcctcagctgcctcctaatcctctggaacaggacctgtggaaccaggccaaagaggacagaatcgaccctctgacactgaaagagatgctggaatccatcagagagacagccgaggaacccctgtctatccagagcctgagattcctgcagctggaactgagcgagatggaagccttcgtgaaccagcacaagagcaacagcatcaagcggcagaccgtgatcaccaccatgaccacactgaagaagaacctggccgacgaggatgccgtgtcttgtctggtgctgggcaccgagaacaaagagctgctggttctggatcccgaggccttcacaatcctggccaagatgtctctgcctagcgtgcccgtgtttctggaagtgtccggccagttcgacgtggaatttcggctggccgctgcctgcagaaacggcaacatctacatcctgcggagggacagcaagcaccccaagtactgtatcgagctgtctgcccagcctgtgggcctgattagagtgcacaaggtgctggtcgtgggcagcacacaggatagcctgcacggctttacccacaagggcaagaaactgtggaccgtgcagatgccagccgccatcctgaccatgaatctgctcgaacagcacagcagaggactgcaggctgttatggcaggactggctaatggcgaagtgcggatctacagagacaaggccctgctgaacgtgatccacacacctgatgccgtgacaagcctgtgcttcggcagatacggcagagaggacaacaccctgatcatgacaacaagaggcggcggactgatcatcaagatcctgaagagaaccgccgtgttcgtggaaggcggatctgaagttggacctcctccagctcaggccatgaagctgaatgtgcccagaaagacccggctgtacgtggaccagacactgagagaaagagaagccggcacagccatgcacagagccttccagactgacctgtacctgctgagactgagagccgccagagcctatctgcaggccctggaatctagcctgtctcctctgagcacaaccgccagagagcctctgaaactgcacgctgtggttcaaggcctgggacctaccttcaagctgaccctgcatctgcagaacaccagcaccacaagaccagtgctgggcctgctcgtgtgcttcctgtacaatgaggccctgtacagcctgccacgggccttttttaaggtgccactgctggtgcccggcctgaactaccctctggaaacctttgtggaaagcctgagcaacaagggcatcagcgacatcatcaaagtgctggtgctgagagagggccagtctgctcctctgctcagcgcccatgtgaatatgcctggctctgaaggcctggcagccgcttaagcgccgtcgac

SEQ ID NO: 12 - DNA, 2425 bp, synthetic construct
  misc_feature 1..2425: note = Construct comprising CMV promoter and COSEQ2-BBS1 nucleotidesequence
  source 1..2425: mol_type = other DNA
actagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgaattcgccaccatggctgccgcctctagctctgactctgatgcatgtggagcagagtctaacgaggccaatagcaagtggctggacgcccactacgatcctatggccaacatccacacattctctgcctgcctggccctggcagacctgcacggcgatggagagtataagctggtggtgggcgacctgggacctggcggccagcagccacggctgaaggtgctgaagggccctctggtcatgacagagtccccactgcccgccctgccagccgccgccgccaccttcctgatggagcagcacgagcctagaaccccagccctggccctggcctctggcccctgcgtgtacgtgtataagaacctgcggccctacttcaagtttagcctgccacagctgccccctaaccctctggagcaggatctgtggaatcaggccaaggaggacaggatcgatcctctgacactgaaggagatgctggagagcatccgggagacagccgaggagccactgagcatccagtccctgagattcctgcagctggagctgtccgagatggaggcctttgtgaaccagcacaagtctaatagcatcaagcgccagaccgtgatcaccacaatgaccacactgaagaagaacctggccgacgaggatgccgtgtcttgtctggtgctgggcacagagaataaggagctgctggtgctggacccagaggccttcaccatcctggccaagatgtctctgccctctgtgcccgtgttcctggaggtgagcggacagttcgacgtggagtttcggctggctgccgcctgcagaaacggcaatatctacatcctgcggagagatagcaagcacccaaagtattgtattgagctgtccgcccagcctgtgggcctgatcagagtgcacaaggtgctggtggtgggcagcacccaggactccctgcacggcttcacacacaagggcaagaagctgtggaccgtgcagatgcccgccgccatcctgaccatgaacctgctggagcagcacagcagaggcctgcaggccgtgatggcaggcctggcaaatggagaggtgaggatctaccgcgacaaggccctgctgaatgtgatccacacccctgatgccgtgacatccctgtgcttcggccggtatggcagagaggataacacactgatcatgaccacacggggcggcggcctgatcatcaagatcctgaagagaacagccgtgtttgtggagggcggctctgaagtgggcccaccccctgcccaggccatgaagctgaatgtgcccaggaagacccgcctgtacgtggaccagacactgagggaaagagaggcaggaacagcaatgcacagggccttccagaccgatctgtacctgctgagactgagagcagcaagagcctatctgcaggccctggagagctccctgtccccactgtctaccacagcaagagaacccctgaagctgcacgcagtggtgcagggcctgggacccaccttcaagctgacactgcacctgcagaacacatccaccacaaggcctgtgctgggcctgctggtgtgcttcctgtacaatgaggccctgtattctctgccacgcgccttctttaaggtgccactgctggtgcccggcctgaactatcccctggagacattcgtggagtccctgtctaataagggcatctctgatatcatcaaggtgctggtgctgagagaaggacagtccgcccctctgctgtctgcccacgtgaatatgccaggcagcgagggcctggctgccgcctgagcggccgcgtcgac

SEQ ID NO: 13 - DNA, 2504 bp, synthetic construct
  misc_feature 1..2504: note = Construct comprising CAG promoter and wild type BBS1 nucleotidesequence
  source 1..2504: mol_type = other DNA
actagttcctggaggggtggagtcgtgacctaggccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggaattccgatggccgctgcgtcctcatcggattccgacgcctgcggagctgagagcaatgaggccaattcgaagtggttggatgcgcactacgacccaatggccaatatccacaccttttctgcctgcctagcgctggcagatttacatggggatggggaatacaagctggtggtaggggaccttggccctggtgggcagcagccccgcctgaaggtgctcaaaggaccactggtgatgaccgaaagcccgctacctgctctgccagctgctgctgccaccttcctcatggagcaacatgagccccggaccccagctctggcacttgcttcaggcccttgtgtctatgtgtataagaatctcagaccctacttcaagttcagcctgccccaattgcctccaaatcctctggaacaagacctttggaaccaggccaaagaggaccgaatcgaccccttaaccctgaaggagatgctggagagcatccgggagacggcagaggagcctttgtccatccagtcactcaggtttctgcagctggagctaagtgaaatggaggcatttgtaaaccaacacaagtccaactccatcaagcggcagacagtcatcaccaccatgaccaccttgaagaagaacctggctgacgaggatgctgtgtcttgcctggtgctgggcaccgagaacaaggagctcctggtgcttgaccccgaggccttcaccattttagccaagatgagccttcccagcgtccccgtcttcctagaggtttctggccagtttgatgttgagttccggcttgccgcggcctgccgcaatggaaacatctatattctgagaagagactccaagcaccccaagtactgcatcgagctgagcgcccagcctgtgggacttatccgggtacacaaggtcctagtggtgggcagcacccaagacagcctgcatggcttcacccacaaggggaagaagctgtggacagtgcagatgcccgcagccatcctgaccatgaacctcctggagcagcattcccggggcctgcaggccgtcatggctgggctggccaatggagaggtccgcatttatcgtgacaaggccctgctcaatgtcatccacaccccggatgcagtgaccagcctttgctttggccggtacgggcgggaggacaacaccctcatcatgaccactcgaggtggtggcctgatcatcaagatcctgaagcgtacagcagtgtttgtagagggaggaagtgaggtgggtcccccaccagcccaggccatgaaactcaatgtgccccgaaagacccggctttacgtggatcagacactgcgagagcgggaggctggcaccgccatgcaccgggccttccagacagacctatacctgctgcgcctacgtgctgcccgcgcctacctgcaggccctcgagtccagcctgagccccctgtccacgacagcccgagagccactcaagctgcacgccgtggttcagggccttggccccacctttaagctcacacttcacctgcagaacacctcaacaacccgtcctgtcctggggctgctggtctgcttcctgtacaacgaggcgctctattccctgccccgggccttcttcaaggtacccttgctggtgccagggctcaactaccccctggagacctttgtggagagtctcagtaacaagggcatctcagacatcatcaaggtgctggtgcttcgagaaggccaaagtgcacccctgctgagtgcccacgtcaacatgcctgggagcgaggggctggcggccgcctgagacttgtcgac

SEQ ID NO: 14 - DNA, 2508 bp, synthetic construct
  misc_feature 1..2508: note = Construct comprising CAG promoter and COSEQ1-BBS1 nucleotidesequence
  source 1..2508: mol_type = other DNA
actagttcctggaggggtggagtcgtgacctaggccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggaattcgccaccatggctgccgccagcagttctgattctgatgcctgtggcgccgagagcaacgaggccaattctaaatggctggacgcccactacgaccccatggccaatatccacacctttagcgcctgtctggccctggctgatcttcatggcgacggcgagtataagctggttgtgggagatcttggacctggcggacagcagcctagactgaaggtgctgaagggccctctcgtgatgacagagtctcctcttcctgctctgcctgccgccgctgccacatttctgatggaacagcacgagcccagaacacccgctctggctcttgcttctggcccttgcgtgtacgtgtacaagaacctgcggccttacttcaagttcagcctgcctcagctgcctcctaatcctctggaacaggacctgtggaaccaggccaaagaggacagaatcgaccctctgacactgaaagagatgctggaatccatcagagagacagccgaggaacccctgtctatccagagcctgagattcctgcagctggaactgagcgagatggaagccttcgtgaaccagcacaagagcaacagcatcaagcggcagaccgtgatcaccaccatgaccacactgaagaagaacctggccgacgaggatgccgtgtcttgtctggtgctgggcaccgagaacaaagagctgctggttctggatcccgaggccttcacaatcctggccaagatgtctctgcctagcgtgcccgtgtttctggaagtgtccggccagttcgacgtggaatttcggctggccgctgcctgcagaaacggcaacatctacatcctgcggagggacagcaagcaccccaagtactgtatcgagctgtctgcccagcctgtgggcctgattagagtgcacaaggtgctggtcgtgggcagcacacaggatagcctgcacggctttacccacaagggcaagaaactgtggaccgtgcagatgccagccgccatcctgaccatgaatctgctcgaacagcacagcagaggactgcaggctgttatggcaggactggctaatggcgaagtgcggatctacagagacaaggccctgctgaacgtgatccacacacctgatgccgtgacaagcctgtgcttcggcagatacggcagagaggacaacaccctgatcatgacaacaagaggcggcggactgatcatcaagatcctgaagagaaccgccgtgttcgtggaaggcggatctgaagttggacctcctccagctcaggccatgaagctgaatgtgcccagaaagacccggctgtacgtggaccagacactgagagaaagagaagccggcacagccatgcacagagccttccagactgacctgtacctgctgagactgagagccgccagagcctatctgcaggccctggaatctagcctgtctcctctgagcacaaccgccagagagcctctgaaactgcacgctgtggttcaaggcctgggacctaccttcaagctgaccctgcatctgcagaacaccagcaccacaagaccagtgctgggcctgctcgtgtgcttcctgtacaatgaggccctgtacagcctgccacgggccttttttaaggtgccactgctggtgcccggcctgaactaccctctggaaacctttgtggaaagcctgagcaacaagggcatcagcgacatcatcaaagtgctggtgctgagagagggccagtctgctcctctgctcagcgcccatgtgaatatgcctggctctgaaggcctggcagccgcttaagcgccgtcgac

SEQ ID NO: 15 - DNA, 2511 bp, synthetic construct
  misc_feature 1..2511: note = Construct comprising CAG promoter and COSEQ2-BBS1 nucleotidesequence
  source 1..2511: mol_type = other DNA
actagttcctggaggggtggagtcgtgacctaggccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggaattcgccaccatggctgccgcctctagctctgactctgatgcatgtggagcagagtctaacgaggccaatagcaagtggctggacgcccactacgatcctatggccaacatccacacattctctgcctgcctggccctggcagacctgcacggcgatggagagtataagctggtggtgggcgacctgggacctggcggccagcagccacggctgaaggtgctgaagggccctctggtcatgacagagtccccactgcccgccctgccagccgccgccgccaccttcctgatggagcagcacgagcctagaaccccagccctggccctggcctctggcccctgcgtgtacgtgtataagaacctgcggccctacttcaagtttagcctgccacagctgccccctaaccctctggagcaggatctgtggaatcaggccaaggaggacaggatcgatcctctgacactgaaggagatgctggagagcatccgggagacagccgaggagccactgagcatccagtccctgagattcctgcagctggagctgtccgagatggaggcctttgtgaaccagcacaagtctaatagcatcaagcgccagaccgtgatcaccacaatgaccacactgaagaagaacctggccgacgaggatgccgtgtcttgtctggtgctgggcacagagaataaggagctgctggtgctggacccagaggccttcaccatcctggccaagatgtctctgccctctgtgcccgtgttcctggaggtgagcggacagttcgacgtggagtttcggctggctgccgcctgcagaaacggcaatatctacatcctgcggagagatagcaagcacccaaagtattgtattgagctgtccgcccagcctgtgggcctgatcagagtgcacaaggtgctggtggtgggcagcacccaggactccctgcacggcttcacacacaagggcaagaagctgtggaccgtgcagatgcccgccgccatcctgaccatgaacctgctggagcagcacagcagaggcctgcaggccgtgatggcaggcctggcaaatggagaggtgaggatctaccgcgacaaggccctgctgaatgtgatccacacccctgatgccgtgacatccctgtgcttcggccggtatggcagagaggataacacactgatcatgaccacacggggcggcggcctgatcatcaagatcctgaagagaacagccgtgtttgtggagggcggctctgaagtgggcccaccccctgcccaggccatgaagctgaatgtgcccaggaagacccgcctgtacgtggaccagacactgagggaaagagaggcaggaacagcaatgcacagggccttccagaccgatctgtacctgctgagactgagagcagcaagagcctatctgcaggccctggagagctccctgtccccactgtctaccacagcaagagaacccctgaagctgcacgcagtggtgcagggcctgggacccaccttcaagctgacactgcacctgcagaacacatccaccacaaggcctgtgctgggcctgctggtgtgcttcctgtacaatgaggccctgtattctctgccacgcgccttctttaaggtgccactgctggtgcccggcctgaactatcccctggagacattcgtggagtccctgtctaataagggcatctctgatatcatcaaggtgctggtgctgagagaaggacagtccgcccctctgctgtctgcccacgtgaatatgccaggcagcgagggcctggctgccgcctgagcggccgcgtcgac

SEQ ID NO: 16 - DNA, 2479 bp, synthetic construct
  misc_feature 1..2479: note = Construct comprising RK promoter and wild type BBS1 nucleotidesequence
  source 1..2479: mol_type = other DNA
gatctcgatcgagttgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccgggctcccaggggcttcccagtggtccccagggaaccctcgacagggccagggcgtctctctcgtccagcaagggcagggacgggccacaggcaagggccctcgatcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcgggcccgggatccaccggggccgcctgcaggtcatcaagcttcgaattccgatggccgctgcgtcctcatcggattccgacgcctgcggagctgagagcaatgaggccaattcgaagtggttggatgcgcactacgacccaatggccaatatccacaccttttctgcctgcctagcgctggcagatttacatggggatggggaatacaagctggtggtaggggaccttggccctggtgggcagcagccccgcctgaaggtgctcaaaggaccactggtgatgaccgaaagcccgctacctgctctgccagctgctgctgccaccttcctcatggagcaacatgagccccggaccccagctctggcacttgcttcaggcccttgtgtctatgtgtataagaatctcagaccctacttcaagttcagcctgccccaattgcctccaaatcctctggaacaagacctttggaaccaggccaaagaggaccgaatcgaccccttaaccctgaaggagatgctggagagcatccgggagacggcagaggagcctttgtccatccagtcactcaggtttctgcagctggagctaagtgaaatggaggcatttgtaaaccaacacaagtccaactccatcaagcggcagacagtcatcaccaccatgaccaccttgaagaagaacctggctgacgaggatgctgtgtcttgcctggtgctgggcaccgagaacaaggagctcctggtgcttgaccccgaggccttcaccattttagccaagatgagccttcccagcgtccccgtcttcctagaggtttctggccagtttgatgttgagttccggcttgccgcggcctgccgcaatggaaacatctatattctgagaagagactccaagcaccccaagtactgcatcgagctgagcgcccagcctgtgggacttatccgggtacacaaggtcctagtggtgggcagcacccaagacagcctgcatggcttcacccacaaggggaagaagctgtggacagtgcagatgcccgcagccatcctgaccatgaacctcctggagcagcattcccggggcctgcaggccgtcatggctgggctggccaatggagaggtccgcatttatcgtgacaaggccctgctcaatgtcatccacaccccggatgcagtgaccagcctttgctttggccggtacgggcgggaggacaacaccctcatcatgaccactcgaggtggtggcctgatcatcaagatcctgaagcgtacagcagtgtttgtagagggaggaagtgaggtgggtcccccaccagcccaggccatgaaactcaatgtgccccgaaagacccggctttacgtggatcagacactgcgagagcgggaggctggcaccgccatgcaccgggccttccagacagacctatacctgctgcgcctacgtgctgcccgcgcctacctgcaggccctcgagtccagcctgagccccctgtccacgacagcccgagagccactcaagctgcacgccgtggttcagggccttggccccacctttaagctcacacttcacctgcagaacacctcaacaacccgtcctgtcctggggctgctggtctgcttcctgtacaacgaggcgctctattccctgccccgggccttcttcaaggtacccttgctggtgccagggctcaactaccccctggagacctttgtggagagtctcagtaacaagggcatctcagacatcatcaaggtgctggtgcttcgagaaggccaaagtgcacccctgctgagtgcccacgtcaacatgcctgggagcgaggggctggcggccgcctgagacttgtcgacctcgagggggggccgcgactctagatcataatcagccataccacatttgtagaggttttacttgctttaaaaaacctcccacacctccccctgaacctgaaacataaaatgaatgcaattgttgttgttaacttgtttat

SEQ ID NO: 17 - DNA, 6278 bp, synthetic construct
  misc_feature 1..6278: note = AAV2/8 construct comprising RK promoter and wild type human BBS1nucleotide sequence
  source 1..6278: mol_type = other DNA
gatctcgatcgagttgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccgggctcccaggggcttcccagtggtccccagggaaccctcgacagggccagggcgtctctctcgtccagcaagggcagggacgggccacaggcaagggccctcgatcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcgggcccgggatccaccggggccgcctgcaggtcatcaagcttcgaattccgatggccgctgcgtcctcatcggattccgacgcctgcggagctgagagcaatgaggccaattcgaagtggttggatgcgcactacgacccaatggccaatatccacaccttttctgcctgcctagcgctggcagatttacatggggatggggaatacaagctggtggtaggggaccttggccctggtgggcagcagccccgcctgaaggtgctcaaaggaccactggtgatgaccgaaagcccgctacctgctctgccagctgctgctgccaccttcctcatggagcaacatgagccccggaccccagctctggcacttgcttcaggcccttgtgtctatgtgtataagaatctcagaccctacttcaagttcagcctgccccaattgcctccaaatcctctggaacaagacctttggaaccaggccaaagaggaccgaatcgaccccttaaccctgaaggagatgctggagagcatccgggagacggcagaggagcctttgtccatccagtcactcaggtttctgcagctggagctaagtgaaatggaggcatttgtaaaccaacacaagtccaactccatcaagcggcagacagtcatcaccaccatgaccaccttgaagaagaacctggctgacgaggatgctgtgtcttgcctggtgctgggcaccgagaacaaggagctcctggtgcttgaccccgaggccttcaccattttagccaagatgagccttcccagcgtccccgtcttcctagaggtttctggccagtttgatgttgagttccggcttgccgcggcctgccgcaatggaaacatctatattctgagaagagactccaagcaccccaagtactgcatcgagctgagcgcccagcctgtgggacttatccgggtacacaaggtcctagtggtgggcagcacccaagacagcctgcatggcttcacccacaaggggaagaagctgtggacagtgcagatgcccgcagccatcctgaccatgaacctcctggagcagcattcccggggcctgcaggccgtcatggctgggctggccaatggagaggtccgcatttatcgtgacaaggccctgctcaatgtcatccacaccccggatgcagtgaccagcctttgctttggccggtacgggcgggaggacaacaccctcatcatgaccactcgaggtggtggcctgatcatcaagatcctgaagcgtacagcagtgtttgtagagggaggaagtgaggtgggtcccccaccagcccaggccatgaaactcaatgtgccccgaaagacccggctttacgtggatcagacactgcgagagcgggaggctggcaccgccatgcaccgggccttccagacagacctatacctgctgcgcctacgtgctgcccgcgcctacctgcaggccctcgagtccagcctgagccccctgtccacgacagcccgagagccactcaagctgcacgccgtggttcagggccttggccccacctttaagctcacacttcacctgcagaacacctcaacaacccgtcctgtcctggggctgctggtctgcttcctgtacaacgaggcgctctattccctgccccgggccttcttcaaggtacccttgctggtgccagggctcaactaccccctggagacctttgtggagagtctcagtaacaagggcatctcagacatcatcaaggtgctggtgcttcgagaaggccaaagtgcacccctgctgagtgcccacgtcaacatgcctgggagcgaggggctggcggccgcctgagacttgtcgacctcgagggggggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcctaggtgagctctggtaccctctagtcaaggatcagtgatggagttggccactccctctctgcgcgctcgctcgctcactgaggccgggcgaccaaaggtcgcccgacgcccgggctttgcccgggcggcctcagtgagcgagcgagcgcgcagctgctgcattaatgaatcggccaacgcgcggggagaggcggtttgcgtattgggcgctcttccgctctagacttaattaaggatcaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctagacttaattaaggatcaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatgcggtgtgaaataccgcacagatgcgtaaggagaaaataccgcatcaggaattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgctgcgcgctcgctcgctcactgaggccgcccgggcaaagcccgggcgtcgggcgacctttggtcgcccggcctcagtgagcgagcgagcgcgcagagagggagtggccaactccatcactgatgtaatacgactcactagtgggca

SEQ ID NO: 18 - DNA, 6401 bp, synthetic construct
  misc_feature 1..6401: note = AAV2/8 construct comprising CMV promoter and wild type BBS1nucleotide sequence
  source 1..6401: mol_type = other DNA
ggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatccgctagcgctaccggactcagatctcgagctcaagcttcgaattccgatggccgctgcgtcctcatcggattccgacgcctgcggagctgagagcaatgaggccaattcgaagtggttggatgcgcactacgacccaatggccaatatccacaccttttctgcctgcctagcgctggcagatttacatggggatggggaatacaagctggtggtaggggaccttggccctggtgggcagcagccccgcctgaaggtgctcaaaggaccactggtgatgaccgaaagcccgctacctgctctgccagctgctgctgccaccttcctcatggagcaacatgagccccggaccccagctctggcacttgcttcaggcccttgtgtctatgtgtataagaatctcagaccctacttcaagttcagcctgccccaattgcctccaaatcctctggaacaagacctttggaaccaggccaaagaggaccgaatcgaccccttaaccctgaaggagatgctggagagcatccgggagacggcagaggagcctttgtccatccagtcactcaggtttctgcagctggagctaagtgaaatggaggcatttgtaaaccaacacaagtccaactccatcaagcggcagacagtcatcaccaccatgaccaccttgaagaagaacctggctgacgaggatgctgtgtcttgcctggtgctgggcaccgagaacaaggagctcctggtgcttgaccccgaggccttcaccattttagccaagatgagccttcccagcgtccccgtcttcctagaggtttctggccagtttgatgttgagttccggcttgccgcggcctgccgcaatggaaacatctatattctgagaagagactccaagcaccccaagtactgcatcgagctgagcgcccagcctgtgggacttatccgggtacacaaggtcctagtggtgggcagcacccaagacagcctgcatggcttcacccacaaggggaagaagctgtggacagtgcagatgcccgcagccatcctgaccatgaacctcctggagcagcattcccggggcctgcaggccgtcatggctgggctggccaatggagaggtccgcatttatcgtgacaaggccctgctcaatgtcatccacaccccggatgcagtgaccagcctttgctttggccggtacgggcgggaggacaacaccctcatcatgaccactcgaggtggtggcctgatcatcaagatcctgaagcgtacagcagtgtttgtagagggaggaagtgaggtgggtcccccaccagcccaggccatgaaactcaatgtgccccgaaagacccggctttacgtggatcagacactgcgagagcgggaggctggcaccgccatgcaccgggccttccagacagacctatacctgctgcgcctacgtgctgcccgcgcctacctgcaggccctcgagtccagcctgagccccctgtccacgacagcccgagagccactcaagctgcacgccgtggttcagggccttggccccacctttaagctcacacttcacctgcagaacacctcaacaacccgtcctgtcctggggctgctggtctgcttcctgtacaacgaggcgctctattccctgccccgggccttcttcaaggtacccttgctggtgccagggctcaactaccccctggagacctttgtggagagtctcagtaacaagggcatctcagacatcatcaaggtgctggtgcttcgagaaggccaaagtgcaccctgctgagtgcccacgtcaacatgcctgggagcgaggggctggcggccgcctgagactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcctaggtgagctctggtaccctctagtcaaggatcagtgatggagttggccactccctctctgcgcgctcgctcgctcactgaggccgggcgaccaaaggtcgcccgacgcccgggctttgcccgggcggcctcagtgagcgagcgagcgcgcagctgctgcattaatgaatcggccaacgcgcggggagaggcggtttgcgtattgggcgctcttccgctctagacttaattaaggatcaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctagacttaattaaggatcaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatgcggtgtgaaataccgcacagatgcgtaaggagaaaataccgcatcaggaattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgctgcgcgctcgctcgctcactgaggccgcccgggcaaagcccgggcgtcgggcgacctttggtcgcccggcctcagtgagcgagcgagcgcgcagagagggagtggccaactccatcactgatgtaatacgactcactagtgggcagatcttcgaatgcatcgcgcgcaccgtacgtctcgaggaattcctgcaggatatcctgcagtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatg

(19) United States
(12) Patent Application Publication
(10) Pub. No.: US 2023/0220420 A1
(43) Pub. Date: Jul. 13, 2023

(54) GENE THERAPY FOR BARDET-BIEDL SYNDROME

(71) Applicant: UCL BUSINESS LTD, London (GB)

(72) Inventors: Philip BEALES, London (GB); Victor HERNANDEZ, London (GB); Sander SMITH, London (GB); Robin ALI, London (GB); Monica FERNANDES FREITAS MARTINS, London (GB)

(21) Appl. No.: 18/047,199

(22) Filed: Oct. 17, 2022

Related U.S. Application Data
(63) Continuation of application No. PCT/GB2021/050932, filed on Apr. 19, 2021.

(30) Foreign Application Priority Data
Jan. 17, 2020 (GB) 2005641.2

(51) Int. Cl. C12N 15/86; A61P 27/02; C07K 14/47

(52) U.S. Cl. CPC C12N 15/86; A61P 27/02; C07K 14/47; C12N 2750/14143; C12N 2750/14171; A61K 48/00

(57) ABSTRACT

There is provided a vector for treating retinal degeneration associated with Bardet-Biedl Syndrome (BBS), wherein the vector comprises a promoter operably linked to a BBS1 gene, wherein the promoter is selected from a rhodopsin kinase (RK) promoter, a cytomegalovirus immediate-early (CMV) promoter and a CAG promoter, and wherein the vector is selected from an AAV2/8 vector, an AAV2/7m8 vector and an AAV9 vector. Also disclosed is a pharmaceutical composition comprising the vector, and use of the vector in a method of treating retinal degeneration associated with BBS comprising administering a therapeutically effective amount of the vector to a patient suffering from BBS, wherein the vector is administered directly to the eye of the patient.

### FIELD OF THE INVENTION

The present invention relates to gene therapy vectors for the treatment of Bardet-Biedl Syndrome. In particular, it relates to the treatment of retinal degeneration associated with Bardet-Biedl Syndrome.

### BACKGROUND TO THE INVENTION

Bardet-Biedl Syndrome (BBS) is an autosomal recessive genetic disorder and is associated with early onset blindness, severe obesity, complex endocrine dysfunction, cognitive impairment and renal failure. Patients born with the inherited Bardet-Biedl syndrome will experience a range of debilitating medical problems, some of which are life-limiting. Affected children will eventually go blind usually beginning in their first decade owing to a failure of the light-sensitive cells at the back of the eye (the retina). Within the first year of life, they will gain an extraordinary amount of body weight which, if unchecked, will progress to life-threatening obesity, diabetes and high blood pressure. Many patients will also develop kidney failure (that may require dialysis treatment and/or kidney transplant) at some point in their lives and most will have some form of learning difficulties. Together these problems will impact adult patients' ability to live independently and most are unemployed. Even when diagnosed early, symptom-based treatments will only manage unpreventable complications such as retinal degeneration and obesity refractory to dietary measures.

So far, 22 genes have been found to be causative in BBS. Many of these gene products interact in multi-subunit complexes. For example, a number of these proteins form a complex called the BBSome. The BBSome is believed to mediate protein trafficking to the primary cilium. The most common gene that is mutated in BBS patients is BBS1. BBS1 gene mutations occur in 42% of patients with BBS. More than 30 mutations in the BBS1 gene have been identified in people with BBS. The human BBS1 gene is located on the long (q) arm of chromosome 11 at position 13. Mutations in the BBS1 gene likely affect the normal formation and function of cilia. Defects in these cell structures disrupt important chemical signalling pathways during development and lead to abnormalities of sensory perception. The human BBS1 gene contains 17 exons and spans approximately 23 kb. Most BBS1 gene mutations are missense or stop mutations and the most common mutation replaces the amino acid methionine with the amino acid arginine at protein position 390 (Met390Arg or M390R). The M390R mutation accounts for approximately 80% of all BBS1 mutations.

The retinal degeneration in BBS is rapidly progressive and devastating to vision, usually causing legal blindness by the age of 20 years. BBS can also present as night blindness and nystagmus in very young children.

So far, treatment of the retinopathy of the eye in mouse models has been attempted using subretinal injection of gene therapy vectors. Such an approach is described in Seo et al., Invest Ophthalmol Vis Sci. 54(9):6118-32 (2013) in which an AAV2/5 vector is used to deliver a mouse Bbs1 gene under the control of a chicken beta actin promoter. However, this study showed that overexpression of BBS1 protein is toxic to the retina in mice. The authors indicated this may be because excess protein cannot incorporate into the normal protein complexes and is free in the cell, interacting with other proteins and hindering their normal function or their normal trafficking. Excess of one protein component may disrupt normal BBSome assembly. Therefore, this study showed that expression of wild-type (WT) BBS1 protein in mice does not necessarily restore normal function.

### SUMMARY OF THE INVENTION

In a first aspect of the invention, there is provided a vector for treating retinal degeneration associated with Bardet-Biedl Syndrome (BBS), wherein the vector comprises a promoter operably linked to a BBS1 gene, wherein the promoter is selected from a rhodopsin kinase (RK) promoter, a cytomegalovirus immediate-early (CMV) promoter and a CAG promoter, and wherein the vector is selected from an AAV2/8 vector, an AAV2/7m8 vector and an AAV9 vector.

The vector is for treating retinal degeneration associated with Bardet-Biedl Syndrome (BBS). In particular, the BBS results from a mutation in the BBS1 gene. Therefore, expression of the BBS1 gene as a result of the vector provides treatment of the BBS.

The vector comprises a BBS1 gene. The BBS1 gene encodes a functional BBS1 protein. The BBS1 gene preferably encodes the human protein, e.g. the wild type human protein. In a patient with BBS, it is a mutation in the BBS1 protein which results in it not being fully functional. This mutated BBS1 protein is responsible for the onset of BBS and the associated retinal degeneration.

The functional BBS1 protein encoded by the BBS1 gene preferably does not contain additional amino acids that are not found in the wild type protein. Any additional amino acids could interfere in the normal functioning of the protein. For example, it is preferred that the functional protein does not comprise a fluorescent protein such as green fluorescent protein (GFP) or mCherry, or tags such such as a FLAG-tag or a polyhistidine-tag.

In particular embodiments, the BBS1 gene has the nucleotide sequence of SEQ ID NO. 1 or has at least 70% sequence identity thereto, and encodes a functional BBS1 protein. In some embodiments, the BBS1 gene has the nucleotide sequence of SEQ ID NO. 1 or has at least 72% sequence identity thereto, and encodes a functional BBS1 protein. In a number of embodiments, the BBS1 gene has the nucleotide sequence of SEQ ID NO. 1 or has at least 74% sequence identity thereto, and encodes a functional BBS1 protein. In other embodiments, the BBS1 gene has the nucleotide sequence of SEQ ID NO. 1 or has at least 76% sequence identity thereto, and encodes a functional BBS1 protein. In various embodiments, the BBS1 gene has the nucleotide sequence of SEQ ID NO. 1 or has at least 78% sequence identity thereto, and encodes a functional BBS1 protein. In particular embodiments, the BBS1 gene has the nucleotide sequence of SEQ ID NO. 1 or has at least 80% sequence identity thereto, and encodes a functional BBS1 protein. In certain embodiments, the BBS1 gene has the nucleotide sequence of SEQ ID NO. 1 or has at least 82% sequence identity thereto, and encodes a functional BBS1 protein. In various embodiments, the BBS1 gene has the nucleotide sequence of SEQ ID NO. 1 or has at least 84% sequence identity thereto, and encodes a functional BBS1 protein. In some embodiments, the BBS1 gene has the nucleotide sequence of SEQ ID NO. 1 or has at least 85% sequence identity thereto, and encodes a functional BBS1 protein. In certain embodiments, the BBS1 gene has the nucleotide sequence of SEQ ID NO. 1 or has at least 86% sequence identity thereto, and encodes a functional BBS1 protein. In a number of embodiments, the BBS1 gene has the nucleotide sequence of SEQ ID NO. 1 or has at least 88% sequence identity thereto, and encodes a functional BBS1 protein. In other embodiments, the BBS1 gene has the nucleotide sequence of SEQ ID NO. 1 or has at least 90% sequence identity thereto, and encodes a functional BBS1 protein. In certain embodiments, the BBS1 gene has the nucleotide sequence of SEQ ID NO. 1 or has at least 92% sequence identity thereto, and encodes a functional BBS1 protein. In a number of embodiments, the BBS1 gene has the nucleotide sequence of SEQ ID NO. 1 or has at least 94% sequence identity thereto, and encodes a functional BBS1 protein. In various embodiments, the BBS1 gene has the nucleotide sequence of SEQ ID NO. 1 or has at least 95% sequence identity thereto, and encodes a functional BBS1 protein. In certain embodiments, the BBS1 gene has the nucleotide sequence of SEQ ID NO. 1 or has at least 96% sequence identity thereto, and encodes a functional BBS1 protein. In some embodiments, the BBS1 gene has the nucleotide sequence of SEQ ID NO. 1 or has at least 97% sequence identity thereto, and encodes a functional BBS1 protein. In a number of embodiments, the BBS1 gene has the nucleotide sequence of SEQ ID NO. 1 or has at least 98% sequence identity thereto, and encodes a functional BBS1 protein. In some embodiments, the BBS1 gene has the nucleotide sequence of SEQ ID NO. 1 or has at least 99% sequence identity thereto, and encodes a functional BBS1 protein. In particular embodiments, the BBS1 gene has the nucleotide sequence of SEQ ID NO. 1.

In the embodiments above, the nucleotide sequence of the BBS1 gene may be codon optimised to maximise expression of the protein. In codon optimisation, the amino acid sequence of the encoded protein remains the same so it will still be functional. It is simply the nucleotide sequence that is modified. SEQ ID NOs. 8 and 9 are codon optimised nucleotide sequences encoding BBS1. These sequences have been found to give an unexpectedly large increase in gene expression.

In particular embodiments, the BBS1 gene has the nucleotide sequence of SEQ ID NO. 8 or has at least 70% sequence identity thereto, and encodes a functional BBS1 protein. In some embodiments, the BBS1 gene has the nucleotide sequence of SEQ ID NO. 8 or has at least 72% sequence identity thereto, and encodes a functional BBS1 protein. In a number of embodiments, the BBS1 gene has the nucleotide sequence of SEQ ID NO. 8 or has at least 74% sequence identity thereto, and encodes a functional BBS1 protein. In other embodiments, the BBS1 gene has the nucleotide sequence of SEQ ID NO. 8 or has at least 76% sequence identity thereto, and encodes a functional BBS1 protein. In various embodiments, the BBS1 gene has the nucleotide sequence of SEQ ID NO. 8 or has at least 78% sequence identity thereto, and encodes a functional BBS1 protein. In particular embodiments, the BBS1 gene has the nucleotide sequence of SEQ ID NO. 8 or has at least 80% sequence identity thereto, and encodes a functional BBS1 protein. In certain embodiments, the BBS1 gene has the nucleotide sequence of SEQ ID NO. 8 or has at least 82% sequence identity thereto, and encodes a functional BBS1 protein. In various embodiments, the BBS1 gene has the nucleotide sequence of SEQ ID NO. 8 or has at least 84% sequence identity thereto, and encodes a functional BBS1 protein. In some embodiments, the BBS1 gene has the nucleotide sequence of SEQ ID NO. 8 or has at least 85% sequence identity thereto, and encodes a functional BBS1 protein. In certain embodiments, the BBS1 gene has the nucleotide sequence of SEQ ID NO. 8 or has at least 86% sequence identity thereto, and encodes a functional BBS1 protein. In a number of embodiments, the BBS1 gene has the nucleotide sequence of SEQ ID NO. 8 or has at least 88% sequence identity thereto, and encodes a functional BBS1 protein. In other embodiments, the BBS1 gene has the nucleotide sequence of SEQ ID NO. 8 or has at least 90% sequence identity thereto, and encodes a functional BBS1 protein. In certain embodiments, the BBS1 gene has the nucleotide sequence of SEQ ID NO. 8 or has at least 92% sequence identity thereto, and encodes a functional BBS1 protein. In a number of embodiments, the BBS1 gene has the nucleotide sequence of SEQ ID NO. 8 or has at least 94% sequence identity thereto, and encodes a functional BBS1 protein. In various embodiments, the BBS1 gene has the nucleotide sequence of SEQ ID NO. 8 or has at least 95% sequence identity thereto, and encodes a functional BBS1 protein. In certain embodiments, the BBS1 gene has the nucleotide sequence of SEQ ID NO. 8 or has at least 96% sequence identity thereto, and encodes a functional BBS1 protein. In some embodiments, the BBS1 gene has the nucleotide sequence of SEQ ID NO. 8 or has at least 97% sequence identity thereto, and encodes a functional BBS1 protein. In a number of embodiments, the BBS1 gene has the nucleotide sequence of SEQ ID NO. 8 or has at least 98% sequence identity thereto, and encodes a functional BBS1 protein. In some embodiments, the BBS1 gene has the nucleotide sequence of SEQ ID NO. 8 or has at least 99% sequence identity thereto, and encodes a functional BBS1 protein. In particular embodiments, the BBS1 gene has the nucleotide sequence of SEQ ID NO. 8.

In particular embodiments, the BBS1 gene has the nucleotide sequence of SEQ ID NO. 9 or has at least 70% sequence identity thereto, and encodes a functional BBS1 protein. In some embodiments, the BBS1 gene has the nucleotide sequence of SEQ ID NO. 9 or has at least 72% sequence identity thereto, and encodes a functional BBS1 protein. In a number of embodiments, the BBS1 gene has the nucleotide sequence of SEQ ID NO. 9 or has at least 74% sequence identity thereto, and encodes a functional BBS1 protein. In other embodiments, the BBS1 gene has the nucleotide sequence of SEQ ID NO. 9 or has at least 76% sequence identity thereto, and encodes a functional BBS1 protein. In various embodiments, the BBS1 gene has the nucleotide sequence of SEQ ID NO. 9 or has at least 78% sequence identity thereto, and encodes a functional BBS1 protein. In particular embodiments, the BBS1 gene has the nucleotide sequence of SEQ ID NO. 9 or has at least 80% sequence identity thereto, and encodes a functional BBS1 protein. In certain embodiments, the BBS1 gene has the nucleotide sequence of SEQ ID NO. 9 or has at least 82% sequence identity thereto, and encodes a functional BBS1 protein. In various embodiments, the BBS1 gene has the nucleotide sequence of SEQ ID NO. 9 or has at least 84% sequence identity thereto, and encodes a functional BBS1 protein. In some embodiments, the BBS1 gene has the nucleotide sequence of SEQ ID NO. 9 or has at least 85% sequence identity thereto, and encodes a functional BBS1 protein. In certain embodiments, the BBS1 gene has the nucleotide sequence of SEQ ID NO. 9 or has at least 86% sequence identity thereto, and encodes a functional BBS1 protein. In a number of embodiments, the BBS1 gene has the nucleotide sequence of SEQ ID NO. 9 or has at least 88% sequence identity thereto, and encodes a functional BBS1 protein. In other embodiments, the BBS1 gene has the nucleotide sequence of SEQ ID NO. 9 or has at least 90% sequence identity thereto, and encodes a functional BBS1 protein. In certain embodiments, the BBS1 gene has the nucleotide sequence of SEQ ID NO. 9 or has at least 92% sequence identity thereto, and encodes a functional BBS1 protein. In a number of embodiments, the BBS1 gene has the nucleotide sequence of SEQ ID NO. 9 or has at least 94% sequence identity thereto, and encodes a functional BBS1 protein. In various embodiments, the BBS1 gene has the nucleotide sequence of SEQ ID NO. 9 or has at least 95% sequence identity thereto, and encodes a functional BBS1 protein. In certain embodiments, the BBS1 gene has the nucleotide sequence of SEQ ID NO. 9 or has at least 96% sequence identity thereto, and encodes a functional BBS1 protein. In some embodiments, the BBS1 gene has the nucleotide sequence of SEQ ID NO. 9 or has at least 97% sequence identity thereto, and encodes a functional BBS1 protein. In a number of embodiments, the BBS1 gene has the nucleotide sequence of SEQ ID NO. 9 or has at least 98% sequence identity thereto, and encodes a functional BBS1 protein. In some embodiments, the BBS1 gene has the nucleotide sequence of SEQ ID NO. 9 or has at least 99% sequence identity thereto, and encodes a functional BBS1 protein. In particular embodiments, the BBS1 gene has the nucleotide sequence of SEQ ID NO. 9.

In various embodiments, the BBS1 gene encodes a functional BBS1 protein having the protein sequence of SEQ ID NO. 2 or at least 80% sequence identity thereto. In some embodiments, the functional BBS1 protein has the protein sequence of SEQ ID NO. 2 or at least 85% sequence identity thereto. In other embodiments, the functional BBS1 protein has the protein sequence of SEQ ID NO. 2 or at least 90% sequence identity thereto. In a number of embodiments, the functional BBS1 protein has the protein sequence of SEQ ID NO. 2 or at least 95% sequence identity thereto. In particular embodiments, the functional BBS1 protein has the protein sequence of SEQ ID NO. 2.

In the description above, the term “identity” is used to refer to the similarity of two sequences. For the purpose of this invention, it is defined here that in order to determine the percent identity of two sequences, the sequences are aligned for optimal comparison purposes (e.g., gaps can be introduced in the sequence of a first sequence for optimal alignment with a second amino or nucleic acid sequence). The nucleotide/amino acid residues at each position are then compared. When a position in the first sequence is occupied by the same amino acid or nucleotide residue as the corresponding position in the second sequence, then the molecules are identical at that position. The percent identity between the two sequences is a function of the number of identical positions shared by the sequences (i.e., % identity=number of identical positions/total number of positions (i.e. overlapping positions)×100). Generally, the two sequences are the same length. A sequence comparison is typically carried out over the entire length of the two sequences being compared.

The skilled person will be aware of the fact that several different computer programs are available to determine the identity between two sequences. For instance, a comparison of sequences and determination of percent identity between two sequences can be accomplished using a mathematical algorithm. In a preferred embodiment, the percent identity between two nucleic acid sequences is determined using the sequence alignment software Clone Manager 9 (Sci-Ed software—www.scied.com) using global DNA alignment; parameters: both strands; scoring matrix: linear (mismatch 2, OpenGap 4, ExtGap 1).

Alternatively, the percent identity between two amino acid or nucleic acid sequences can be determined using the Needleman and Wunsch (1970) algorithm which has been incorporated into the GAP program in the Accelrys GCG software package (available at http://www.accelrys.com/products/gcg/), using either a Blosum 62 matrix or a PAM250 matrix, and a gap weight of 16, 14, 12, 10, 8, 6, or 4 and a length weight of 1, 2, 3, 4, 5, or 6. A further method to assess the percent identity between two amino acid or nucleic acid sequences can be to use the BLAST sequence comparison tool available on the National Center for Biotechnology Information (NCBI) website (www.blast.ncbi.nlm.nih.gov), for example using BLASTn for nucleotide sequences or BLASTp for amino acid sequences using the default parameters.

The BBS1 gene encodes a ‘functional’ protein. This means that the protein, when expressed, has the same function and activity as the wild type human protein. This could easily be determined by one skilled in the art. The protein encoded by the BBS1 gene may be the wild type human protein. The wild type human sequence of the BBS1 protein is well known to those skilled in the art. For example, it can be found on the publically accessible databases of the National Center for Biotechnology Information. Further, the nucleotide sequences which encode this protein (and which would be contained in the vector) could readily be found or determined by a person skilled in the art, for example, using the genetic code which correlates particular nucleotide codons with particular amino acids.

The promoter is selected from a rhodopsin kinase (RK) promoter, a cytomegalovirus immediate-early (CMV) promoter and a CAG promoter. These promoters are well known to one skilled in the art. It has been found that these promoters provide advantageous results when used to express the BBS1 gene in cells of the eye.

Examples of the sequences of these promoters are given as SEQ ID NO. 3, SEQ ID NO. 4, SEQ ID NO. 5, SEQ ID NO. 6 and SEQ ID NO. 7. Therefore, in some embodiments, the promoter has a sequence selected from SEQ ID NO. 3, SEQ ID NO. 4, SEQ ID NO. 5, SEQ ID NO. 6 and SEQ ID NO. 7.

In some embodiments, the promoter is a rhodopsin kinase (RK) promoter. Preferably, it is a human rhodopsin kinase (RK) promoter which may optionally have the nucleotide sequence of SEQ ID NO. 7. For example, the vector may comprise the sequence of SEQ ID NO. 16.

In some embodiments, the promoter is a CAG promoter which may optionally have the nucleotide sequence of SEQ ID NO. 3 or SEQ ID NO. 4. For example, the vector may comprise the sequence of one of SEQ ID NOs. 13, 14 and 15.

In various embodiments, the promoter is a CMV promoter which may optionally have the nucleotide sequence of SEQ ID NO. 5 or SEQ ID NO. 6. For example, the vector may comprise the sequence of one of SEQ ID NOs. 10, 11 and 12.

The vector described above is for treating retinal degeneration associated with Bardet-Biedl Syndrome (BBS). To achieve this, the vector can transduce cells of the eye such as retinal photoreceptors and/or retinal pigmented epithelial cells. In particular, when delivered subretinally, the vector can transduce retinal photoreceptors (outer nuclear layer, inner and outer segment) and retinal pigmented epithelial cells. Alternatively, the vector can be delivered intravitreally. This can be used to target the internal retinal layers.

The vector is selected from an AAV2/8 vector, an AAV2/7m8 vector and an AAV9 vector. An AAV2/8 vector is an AAV2 vector which has been pseudotyped with the capsid proteins from AAV8. Such vectors are described in WO 2005/033321. An AAV2/7m8 vector is an AAV2 vector which has been pseudotyped with the capsid proteins from AAV serotype 7m8. AAV 7m8 vectors are described in Biotechnol Bioeng. 2016 December; 113(12):2712-2724. doi: 10.1002/bit.26031. Mol Ther. 2018 May 2; 26(5):1343-1353. doi: 10.1016/j.ymthe.2018.02.027. J Struct Biol. 2020 Feb. 1; 209(2):107433. doi: 10.1016/j.jsb.2019.107433. In some embodiments, the vector is an AAV2/8 vector. In other embodiments, the vector is an AAV2/7m8 vector. The AAV2/7m8 vector is particularly suitable for intravitreal administration. In various embodiments, the vector is an AAV9 vector.

In some embodiments, the vector is an AAV2/8 vector, the promoter is a rhodopsin kinase (RK) promoter, and the BBS1 gene encodes a functional human BBS1 protein.

In various embodiments, the vector is an AAV2/8 vector, the promoter is a cytomegalovirus immediate-early (CMV) promoter, and the BBS1 gene encodes a functional human BBS1 protein.

In certain embodiments, the vector is an AAV2/8 vector, the promoter is a CAG promoter, and the BBS1 gene encodes a functional human BBS1 protein.

In some embodiments, the vector is an AAV2/7m8 vector, the promoter is a rhodopsin kinase (RK) promoter, and the BBS1 gene encodes a functional human BBS1 protein.

In various embodiments, the vector is an AAV2/7m8 vector, the promoter is a cytomegalovirus immediate-early (CMV) promoter, and the BBS1 gene encodes a functional human BBS1 protein.

In certain embodiments, the vector is an AAV2/7m8 vector, the promoter is a CAG promoter, and the BBS1 gene encodes a functional human BBS1 protein.

In particular embodiments, the vector is an AAV9 vector, the promoter is a rhodopsin kinase (RK) promoter, and the BBS1 gene encodes a functional human BBS1 protein.

In a number embodiments, the vector is an AAV9 vector, the promoter is a cytomegalovirus immediate-early (CMV) promoter, and the BBS1 gene encodes a functional human BBS1 protein.

In several embodiments, the vector is an AAV9 vector, the promoter is a CAG promoter, and the BBS1 gene encodes a functional human BBS1 protein.

The adeno-associated viral vector may be a recombinant adeno-associated viral (rAAV) vector. AAV is a member of the family Parvoviridae which is described in Kenneth I. Berns, “Parvoviridae: The Viruses and Their Replication,” Chapter 69 in Fields Virology (3d Ed. 1996).

The genomic organization of all known AAV serotypes is very similar. The genome of AAV is a linear, single-stranded DNA molecule that is less than about 5,000 nucleotides (nt) in length. Inverted terminal repeats (ITRs) flank the unique coding nucleotide sequences for the non-structural replication (Rep) proteins and the structural (VP) proteins. The VP proteins (VP1, -2 and -3) form the capsid. The terminal 145 nt are self-complementary and are organized so that an energetically stable intramolecular duplex forming a T-shaped hairpin may be formed. These hairpin structures function as an origin for viral DNA replication, serving as primers for the cellular DNA polymerase complex. Following wild type (wt) AAV infection in mammalian cells the Rep genes (i.e. encoding Rep78 and Rep52 proteins) are expressed from the P5 promoter and the P19 promoter, respectively, and both Rep proteins have a function in the replication of the viral genome. A splicing event in the Rep ORF results in the expression of actually four Rep proteins (i.e. Rep78, Rep68, Rep52 and Rep40). However, it has been shown that the unspliced mRNA, encoding Rep78 and Rep52 proteins, in mammalian cells are sufficient for AAV vector production. Also in insect cells the Rep78 and Rep52 proteins suffice for AAV vector production.

In an AAV suitable for use as a gene therapy vector, the vector genome typically comprises a nucleic acid (e.g. a BBS1 gene) to be packaged for delivery to a target cell. According to this particular embodiment, the heterologous nucleotide sequence is located between the viral ITRs at either end of the vector genome. In further preferred embodiments, the parvovirus (e.g. AAV) cap genes and parvovirus (e.g. AAV) rep genes are deleted from the template genome (and thus from the virion DNA produced therefrom). This configuration maximizes the size of the nucleic acid sequence(s) that can be carried by the parvovirus capsid.

According to this particular embodiment, the nucleic acid is located between the viral ITRs at either end of the substrate. It is possible for a parvoviral genome to function with only one ITR. Thus, in a gene therapy vector based on a parvovirus, the vector genome is flanked by at least one ITR, but, more typically, by two AAV ITRs (generally with one either side of the vector genome, i.e. one at the 5′ end and one at the 3′ end). There may be intervening sequences between the nucleic acid in the vector genome and one or more of the ITRs.

Generally, the BBS1 gene will be incorporated into a parvoviral genome located between two regular ITRs or located on either side of an ITR engineered with two D regions.

In one aspect, the invention provides a pharmaceutical composition comprising a vector as described above and one or more pharmaceutically acceptable excipients. The one or more excipients include carriers, diluents and/or other medicinal agents, pharmaceutical agents or adjuvants, etc.

The invention also provides a method of treating retinal degeneration associated with Bardet-Biedl Syndrome (BBS) comprising administering a therapeutically effective amount of a vector as described above to a patient suffering from BBS. Preferably, the patient is human. The patient will have a mutation which causes the BBS1 protein not to be fully functional. This may be biallelic mutations.

When the retinal degeneration is “treated” in the above method, this means that the retinal degeneration associated with BBS is slowed down so that the rate of deterioration of a patient's eyesight is not as fast as before treatment. In some embodiments, the vector may stop any further retinal degeneration so that there is no further deterioration in a patient's eyesight. Whilst preferably the eyesight of a patient will improve following treatment with the vector, this may not occur in all cases.

A “therapeutically effective amount” refers to an amount effective, at dosages and for periods of time necessary, to achieve the desired therapeutic result, such as raising the level of functional protein in a subject (so as to lead to a level sufficient to ameliorate the pathologies associated with BBS).

The method of treatment causes an increase in the level of functional protein in the subject. In some embodiments, the method of treatment causes an increase in the level of functional protein to about a normal level (i.e. the level found in a normal healthy subject). In one embodiment, the method of treatment causes an increase in the level of functional protein to, at most, normal levels.

The vector may be administered in any suitable way so as to allow expression of the BBS1 gene in the cells of the eye. In particular embodiments, a single administration of the vector can be used to provide gene expression to ameliorate the pathologies associated with BBS. The vector should be administered directly to the eye. For example, this may be subretinal or intravitreal administration such as by subretinal injection or intravitreal injection. In subretinal administration/injection, the vector is delivered into the subretinal space between retinal pigment epithelium (RPE) cells and photoreceptors. In the subretinal space, administered material comes into direct contact with the plasma membrane of the photoreceptor, and RPE cells and subretinal blebs. This makes it an excellent site for drug delivery in patients with retinal degeneration associated with BBS. The vector may be administered at a single point in time. For example, a single injection may be given. In some embodiments, one or more further administrations of the vector can be given.

Further, the invention provides the vector described above for use in therapy, for example, in the treatment of retinal degeneration associated with Bardet-Biedl Syndrome (BBS).

In addition, the invention provides the use of the vector as described above in the manufacture of a medicament for treating retinal degeneration associated with Bardet-Biedl Syndrome (BBS).

All patent and literature references cited in the present specification are hereby incorporated by reference in their entirety.

### BRIEF DESCRIPTION OF THE DRAWINGS

The invention will now be described in detail by way of example only with reference to the figures which are as follows:

FIG. 1: BBS1 mutation leads to degeneration of the photoreceptors layers in Bbs1^{M390R/M390R} mice. Histological analysis show the degeneration of the outer nuclear layer and photoreceptor numbers 6 month after birth. This degeneration is also observed by the reduction of both photopic and scotopic amplitude at 6 months of age of Bbs1^{M390R/M390R} mice compare with wild-type littermates.

FIGS. 2A-2C: AAV2/8.RK.hBBS1 supplementation of BBS1 only in photoreceptors is not a long term efficient therapeutic strategy in Bbs1^{M390R/M390R} mice. FIG. 2A shows that delivery of the AAV2/8.RK.hBBS1 vector achieve a small short term recovery of the ERG responses 3 months after delivery but is not able to sustain the recovery long-term when compared with the contralateral control eyes as seen after 11 months of treatment. FIG. 2A shows scotopic a-wave amplitude (uV). FIG. 2B shows scotopic b-wave amplitude (uV). FIG. 2C shows photopic b-wave amplitude (uV). Blue points indicate responses of eyes injected with AAV2/8.RK.hBBS1 and red points indicate responses from control contralateral eyes. Error bars are Standard Deviation (SD).

FIGS. 3A-3C: Subretinal delivery of AAV2/8.CMV.hBBS1 results in variability in its efficiency to rescue retinal degeneration in Bbs1^{M390R/M390R} mice. We found there is high variability in the ERG responses of animal injected with the AAV2/8.CMV.hBBS1 vector. Statistical analysis show rescues at different light stimulus for the scotopic and photopic b-wave for P7-9 and P30 injected retinae up to 6 months post injection. FIG. 3A shows scotopic a-wave amplitude (uV). FIG. 3B shows scotopic b-wave amplitude (uV). FIG. 3C shows photopic b-wave amplitude (uV). Blue points indicate responses of eyes injected with AAV2/8.CMV.hBBS1 and red points indicate responses from control contralateral eyes. Error bars are Standard Deviation (SD).

FIGS. 4A and 4B: Subretinal delivery of AAV2/8.CMV.hBBS1 completely halts retinal degeneration in Bbs1^{M390R/M390R} in some treated animals for a year. FIG. 4A shows that half of the P7-9 treated retinae achieve complete retinal degeneration rescue up to 11 month after injection of a- and b-wave scotopic and b-wave photopic responses up to 11 months after treatment for all light intensity tested. Blue points indicate responses of eyes injected with AAV2/8.RK.hBBS1 and red points indicate responses from control contralateral eyes. Error bars are Standard Deviation (SD). FIG. 4B shows histological analysis of treated retinae show complete recovery of the outer nuclear layer with maintenance of a nuclei in the layer. Controlateral control untreated eyes show complete lost of all photoreceptors and cell nuclei.

FIG. 5: Overexpression of BBS1 is not toxic in wild-type (WT) or in Bbs1^{M390R/M390R} mice. We tested overexpression of BBS1 transgene in the photoreceptors of wild-type P30 animals with low and high doses of AAV2/8.RK-hBBS1 and photopic and Scotopic ERG responses were measured. TUNEL staining was carried out to look for any increase in apoptosis in injected eyes. No TUNEL staining was seen in any of the sections tested from three controls and three injected retinae suggesting no apoptosis is occurring. A western blot was carried out using total retinae protein extracts. The top panel shows the blot probed with an antibody specific to the activated form of caspase 3 (marked with an arrow). The second panel shows a β-Actin loading control. Titer injected in: A; 1×10¹¹, B; 8×10¹¹ and C; 4×10¹² vg/ml.

### DETAILED DESCRIPTION OF THE INVENTION

The inventors have developed an ocular gene therapy to halt and further prevent the retinal degeneration phenotype associated with BBS1 deficiency. The inventors found that although Bbs1^{M390R/M390R} mice presented mainly a photoreceptor degeneration of the outer nuclear layer as seen in patients, supplementation of human BBS1 only in photoreceptors cells alone was not always efficient or sufficient to prevent retinal degeneration. The inventors then explored the therapeutic effects of delivering BBS1 to also target the retinal pigmented epithelium (RPE) cells, highly ciliated and crucial for photoreceptor survival. Expression of the BBS1 transgene in both photoreceptors and RPE was able to prevent completely the degeneration of the photoreceptor layers. Variability in the treatment effect between different treated individuals was observed. The complexity of the function of the BBS1 gene as part of a multi-complex unit might explain the variability on the treatment effect observed.

The inventors demonstrated that supplementing BBS1 in photoreceptors and RPE leads to functional and cellular rescue of the retinal degeneration in Bbs1^{M390R/M390R} animals.

### Materials and Methods

A construct has been produced where human BBS1 cDNA (SEQ ID NO. 1—NM_024649.4) has been cloned under the control of the human rhodopsin kinase (RK) promoter in an AAV2 viral plasmid. For virus production, usual methods were used to produce an AAV2/8 virus. 4000 cm² of HEK293T cell monolayer cells were transfected with the RK-BBS1-AAV-ITR containing plasmid, AAV2 Rep-Cap plasmid and the helper plasmid. Once showing cytopathic effects, cells were harvested and lysed to release the virus. The adeno-associated virus was purified by Sephacryl 5300 column followed by anion exchange chromatography using a POROS 50 HQ column. The final product was titered by quantitative real-time PCR using AAV specific probe and a SV-40 probe as a standard.

### Virus Administration and Titer

Time matings were prepared between Bbs1^{M390R/+} males and Bbs1^{M390R/-} females. Pups were genotyped for Bbs1 genotype. The adenoviral-associated vector was given via subretinal unilaterally injection in P7-9 and P30 1×10¹² vg/ml (vector genomes/ml). The inventors injected 2 different groups of animals; Bbs1^{M390R/M390R} animals, wild-type. Contralateral eyes serve as controls for each group. A total of n=10 animals/group were used. Treated animals do not show any physical or behaviour distress after 12 months post-injection.

### Results

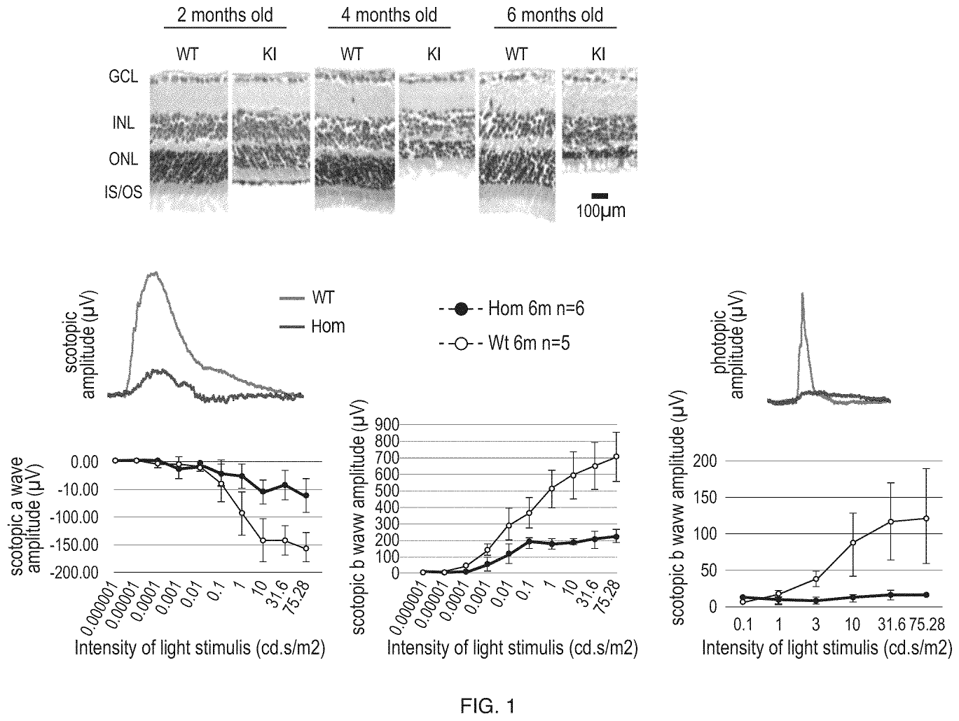

FIG. 1: BBS1 mutation leads to degeneration of the photoreceptors layers in Bbs1^{M390R/M390R} mice. Histological analysis show the degeneration of the outer nuclear layer and photoreceptor numbers 6 month after birth. This degeneration is also observed by the reduction of both photopic and scotopic amplitude at 6 months of age of Bbs1^{M390R/M390R} mice compare with wild-type littermates.

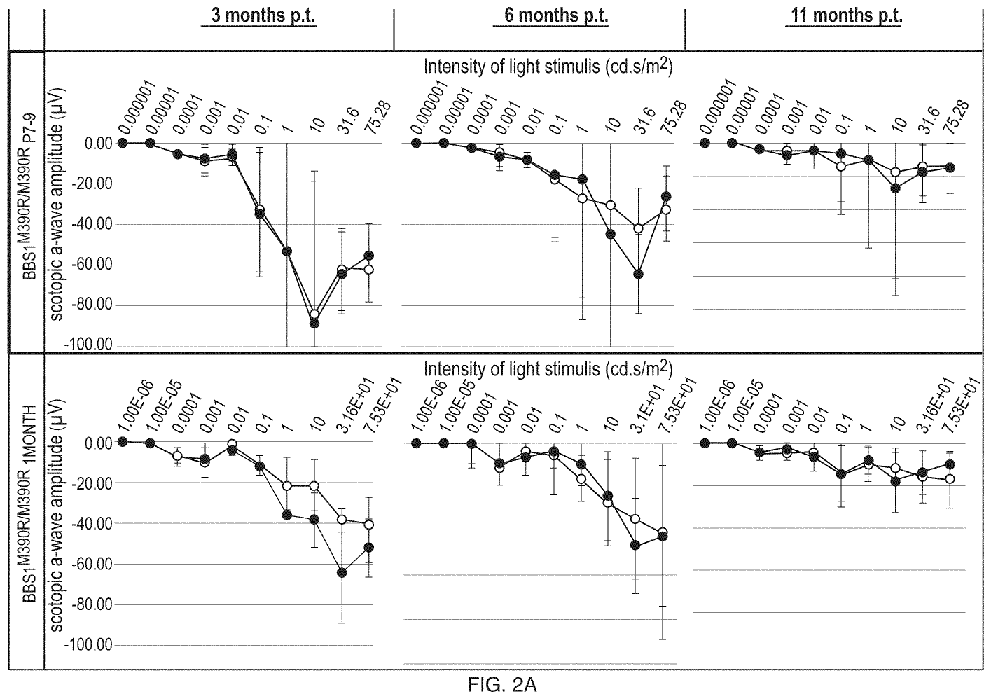

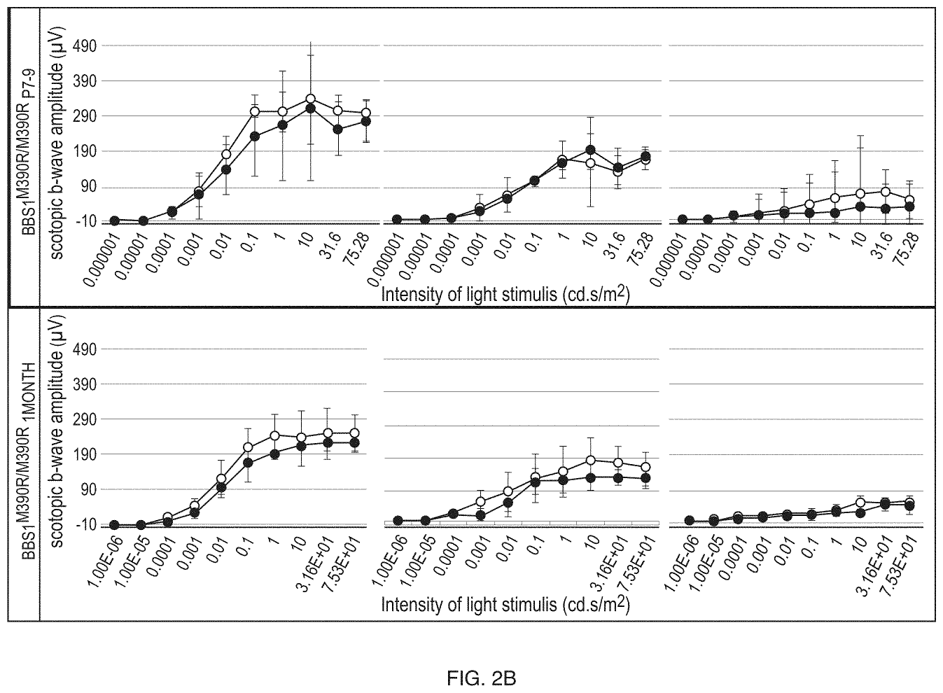

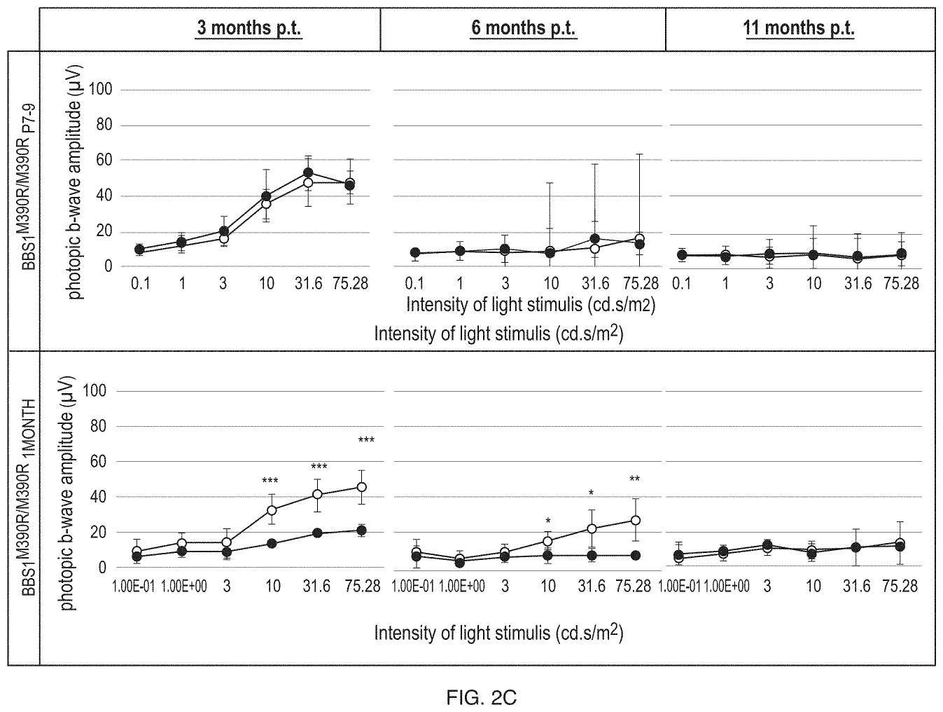

FIGS. 2A-2C: AAV2/8.RK.hBBS1 supplementation of BBS1 only in photoreceptors is not a long term efficient therapeutic strategy in Bbs1^{M390R/M390R} mice. Delivery of the AAV2/8.RK.hBBS1 vector achieves a small short term recovery of the ERG responses 3 months after delivery but is not able to sustain the recovery long-term when compared with the contralateral control eyes as seen after 11 months of treatment. Blue points indicate responses of eyes injected with AAV2/8.RK.hBBS1 and red points indicate responses from control contralateral eyes. Error bars are Standard Deviation (SD).

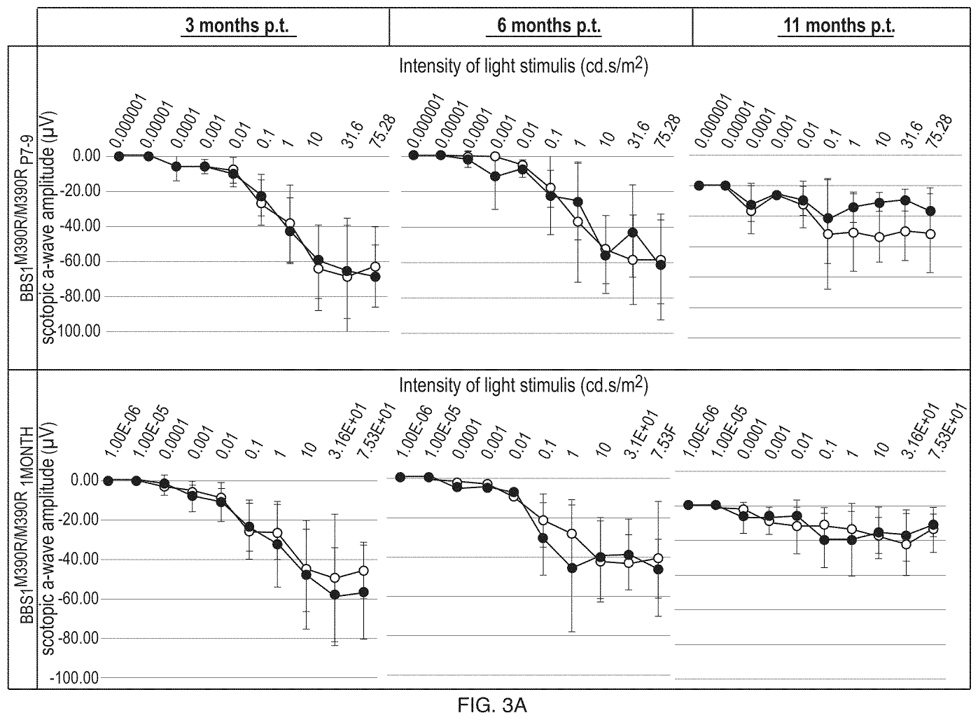

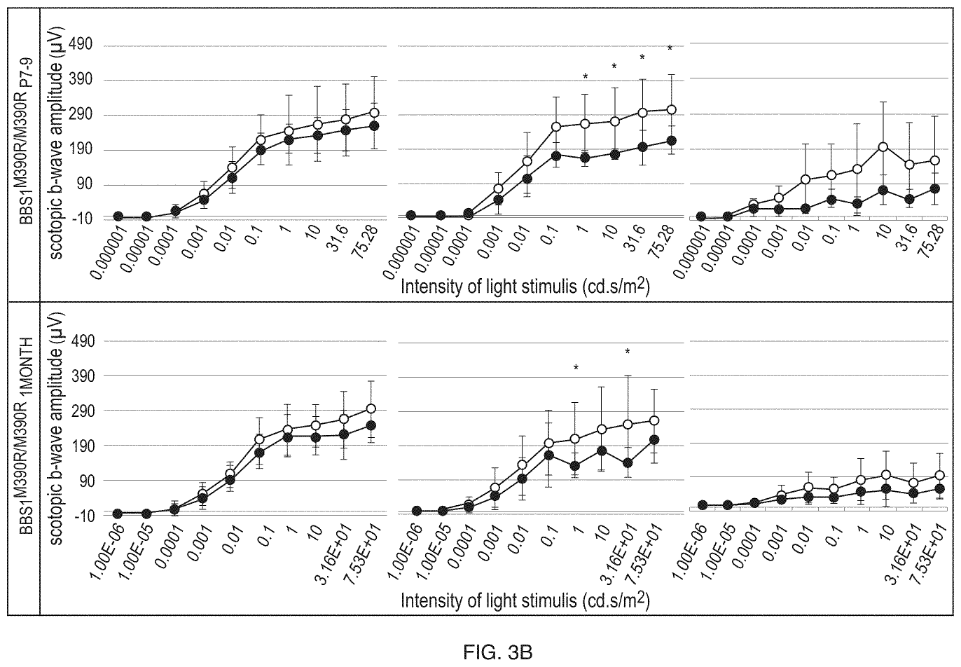

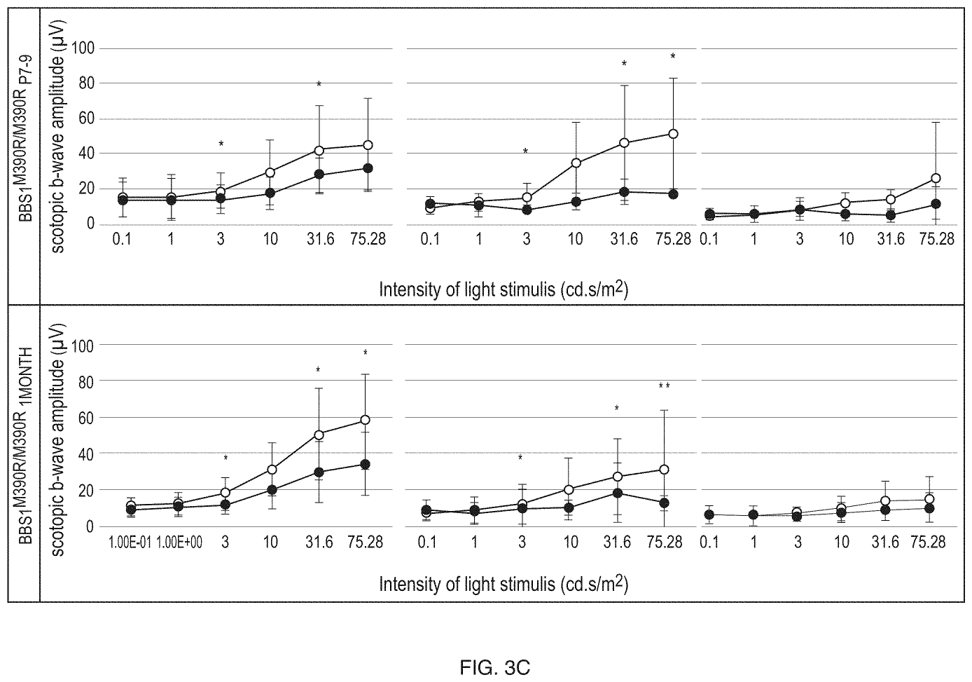

FIGS. 3A-3C: Subretinal delivery of AAV2/8.CMV.hBBS1 results in variability in its efficiency to rescue retinal degeneration in Bbs1^{M390R/M390R} mice. We found there is high variability in the ERG responses of animal injected with the AAV2/8.CMV.hBBS1 vector. Statistical analysis show rescues at different light stimulus for the scotopic and photopic b-wave for P7-9 and P30 injected retinae up to 6 months post injection. Blue points indicate responses of eyes injected with AAV2/8.CMV.hBBS1 and red points indicate responses from control contralateral eyes. Error bars are Standard Deviation (SD).

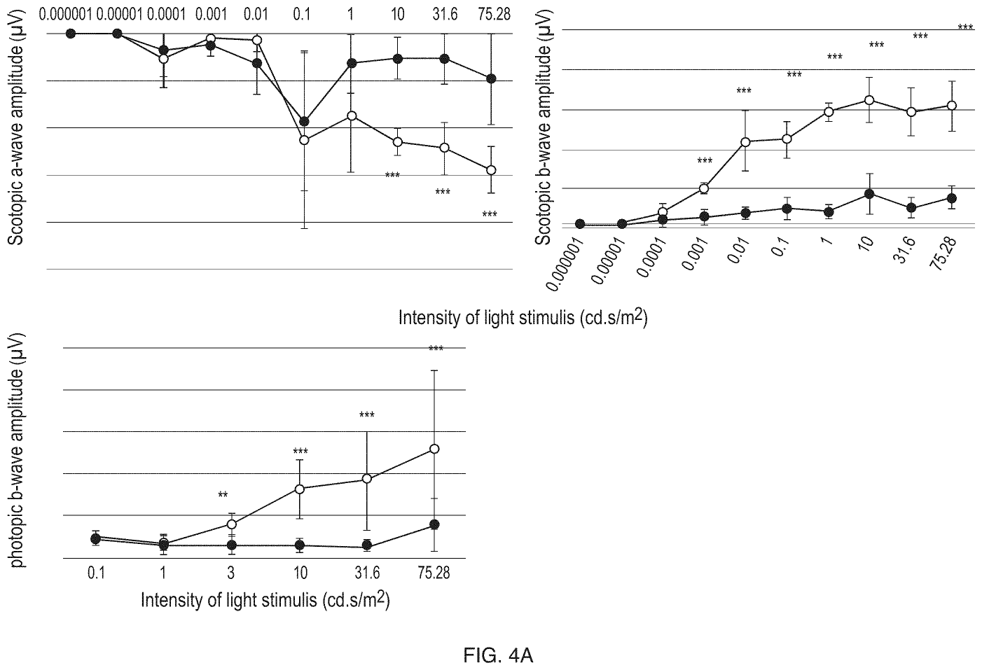

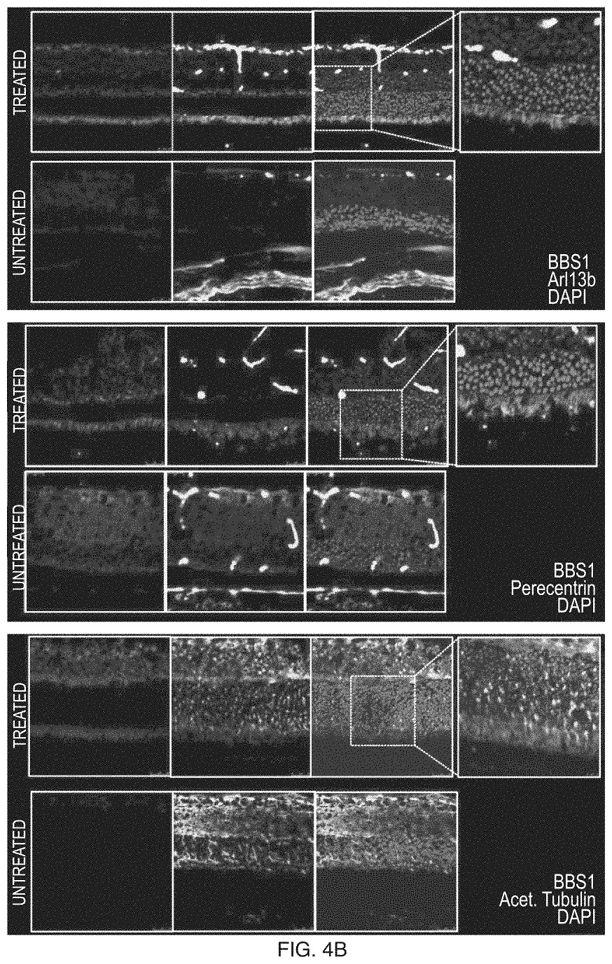

FIGS. 4A and 4B: Subretinal delivery of AAV2/8.CMV.hBBS1 completely halts retinal degeneration in Bbs1^{M390R/M390R} in some treated animals for a year. Half of the P7-9 treated retinae achieve complete retinal degeneration rescue up to 11 month after injection of a- and b-wave scototopic and b-wave photopic responses up to 11 months after treatment for all light intensity tested. Blue points indicate responses of eyes injected with AAV2/8.RK.hBBS1 and red points indicate responses from control contralateral eyes. Error bars are Standard Deviation (SD). Histological analysis of treated retinae show complete recovery of the outer nuclear layer with maintenance of a nuclei in the layer. Controlateral control untreated eyes show complete loss of all photoreceptors and cell nuclei.

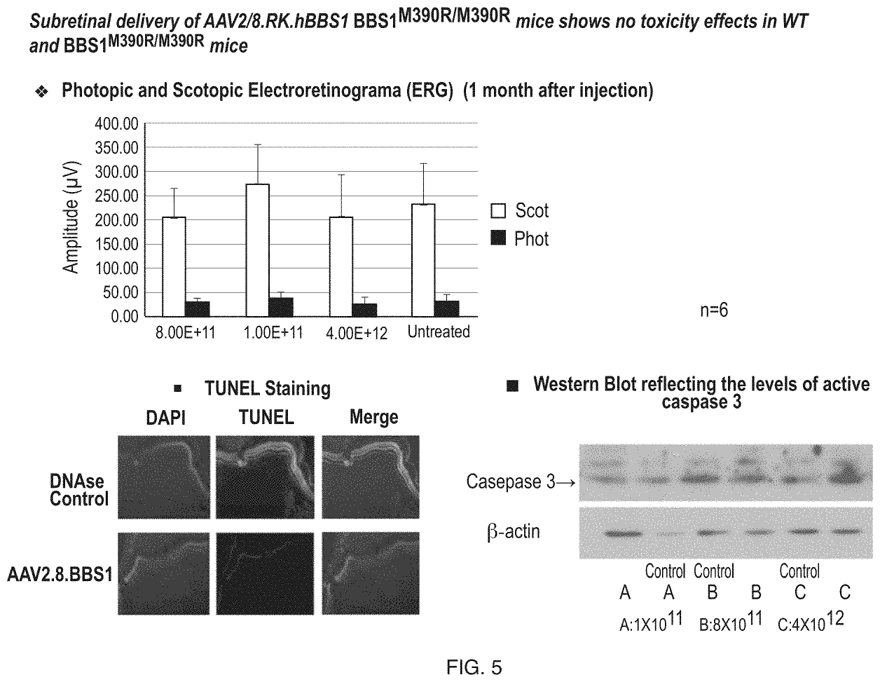

FIG. 5: Overexpression of BBS1 is not toxic in wild-type (WT) or in Bbs1^{M390R/M390R} mice. We tested overexpression of BBS1 transgene in the photoreceptors of wild-type P30 animals with low and high doses of AAV2/8.RK-hBBS1 and photopic and Scotopic ERG responses were measured. TUNEL staining was carried out to look for any increase in apoptosis in injected eyes. No TUNEL staining was seen in any of the sections tested from three controls and three injected retinae suggesting no apoptosis is occurring. A western blot was carried out using total retinae protein extracts. The top panel shows the blot probed with an antibody specific to the activated form of caspase 3 (marked with an arrow). The second panel shows a β-Actin loading control. Titer injected in: A; 1×10¹¹, B; 8×10¹¹ and C; 4×10¹² vg/ml.

These results demonstrate that the vector has advantageous properties compared to the vector used in Seo et al. (Invest Ophthalmol Vis Sci. 54(9):6118-32 (2013)) in which overexpression of BBS1 protein was shown to be toxic to the retina in mice.

### SEQUENCES

SEQ ID NO. 1—Human Bardet-Biedl syndrome 1 (BBS1) nucleotide sequence (WT), cDNA (NM_024649.4)

SEQ ID NO. 2—Human BBS1 full protein sequence (Q8NFJ9)

SEQ ID NO. 3—CAG promoter sequence

SEQ ID NO. 4—Alternative CAG promoter sequence

SEQ ID NO. 5—Cytomegalovirus (CMV) immediate-early promoter sequence

SEQ ID NO. 6—Alternative CMV promoter sequence

SEQ ID NO. 7—Rhodopsin kinase promoter sequence

SEQ ID NO. 8—Codon optimised nucleotide sequence encoding human BBS1 protein (referred to as COSEQ1-BBS1)

SEQ ID NO. 9—Codon optimised nucleotide sequence encoding human BBS1 protein (referred to as COSEQ2-BBS1)

SEQ ID NO. 10—Construct comprising CMV promoter (nt 52-256) and wild type BBS1 nucleotide sequence (nt 324-2108)

SEQ ID NO. 11—Construct comprising CMV promoter (nt 367-570) and COSEQ1-BBS1 nucleotide sequence (nt 630-2411)

SEQ ID NO. 12—Construct comprising CMV promoter (nt 367-570) and COSEQ2-BBS1 nucleotide sequence (nt 630-2411)

SEQ ID NO. 13—Construct comprising CAG promoter (nt 35-562) and wild type BBS1 nucleotide sequence (nt 712-2493)

SEQ ID NO. 14—Construct comprising CAG promoter (nt 35-562) and COSEQ1-BBS1 nucleotide sequence (nt 716-2497)

SEQ ID NO. 15—Construct comprising CAG promoter (nt 35-562) and COSEQ2-BBS1 nucleotide sequence (nt 716-2497)

SEQ ID NO. 16—Construct comprising RK promoter (nt 16-255) and wild type human BBS1 nucleotide sequence (nt 546-2330)

SEQ ID NO. 17—AAV2/8 construct comprising RK promoter (nt 16-255) and wild type human BBS1 nucleotide sequence (nt 546-2330)

SEQ ID NO. 18—AAV2/8 construct comprising CMV promoter (nt 52-256) and wild type human BBS1 nucleotide sequence (nt 324-2108)

1. A vector for treating retinal degeneration associated with Bardet-Biedl Syndrome (BBS), wherein the vector comprises a promoter operably linked to a BBS1 gene, wherein the promoter is selected from a rhodopsin kinase (RK) promoter, a cytomegalovirus immediate-early (CMV) promoter and a CAG promoter, and wherein the vector is selected from an AAV2/8 vector, an AAV2/7m8 vector and an AAV9 vector.

2. (canceled)

3. A vector according to claim 1, wherein the promoter is a human RK promoter.

4. A vector according to claim 3, wherein the RK promoter comprises the sequence of SEQ ID NO. 7.

5. A vector according to claim 1, wherein the promoter is a CMV promoter.

6. A vector according to claim 5, wherein the CMV promoter comprises a nucleotide sequence selected from SEQ ID NO. 5 and SEQ ID NO. 6.

7. A vector according to claim 1, wherein the promoter is a CAG promoter.

8. A vector according to claim 7, wherein the CAG promoter comprises a nucleotide sequence selected from SEQ ID NO. 3 and SEQ ID NO. 4.

9.-11. (canceled)

12. A vector according to claim 1, wherein the BBS1 gene encodes a functional human BBS1 protein comprising the amino acid sequence of SEQ ID NO. 2 or an amino acid sequence with at least 80% sequence identity thereto.

13. A vector according to claim 1, wherein the BBS1 gene encodes a wild type human BBS1 protein.

14. A vector according to claim 1, wherein the BBS1 gene comprises the nucleotide sequence of SEQ ID NO. 1 or a nucleotide sequence with at least 70% sequence identity thereto, and encodes a functional human BBS1 protein.

15. A vector according to claim 14, wherein the BBS1 gene comprises the nucleotide sequence of SEQ ID NO. 1.

16. A vector according to of claim 1, wherein the BBS1 gene comprises the nucleotide sequence of SEQ ID NO. 8 or 9.

17. A vector according to claim 1, wherein:
1) the BBS1 gene comprises the nucleotide sequence of SEQ ID NO: 1, and the promoter is a human RK promoter;
2) the BBS1 gene comprises the nucleotide sequence of SEQ ID NO. 8, and the promoter is a human RK promoter;
3) the BBS1 gene comprises the nucleotide sequence of SEQ ID NO. 9, and the promoter is a human RK promoter;
4) the BBS1 gene comprises the nucleotide sequence of SEQ ID NO. 1, and the promoter is a CMV promoter;
5) the BBS1 gene comprises the nucleotide sequence of SEQ ID NO. 8, and the promoter is a CMV promoter;
6) the BBS1 gene comprises the nucleotide sequence of SEQ ID NO. 9, and the promoter is a CMV promoter;
7) the BBS1 gene comprises the nucleotide sequence of SEQ ID NO. 1, and the promoter is a CAG promoter;
8) the BB gene comprises the nucleotide sequence of SEQ ID NO. 8, and the promoter is a CAG promoter; or
9) the BB gene comprises the nucleotide sequence of SEQ ID NO. 9, and the promoter is a CAG promoter.

18.-19. (canceled)

20. A vector according to claim 1, wherein:
1) the vector is an AAV2/8 vector, the promoter is a rhodopsin kinase (RK) promoter, and the BBS1 gene encodes a functional human BBS1 protein;
2) the vector is an AAV2/8 vector, the promoter is a cytomegalovirus immediate-early (CMV) promoter, and the BB S1 gene encodes a functional human BBS1 protein;
3) the vector is an AAV2/8 vector, the promoter is a CAG promoter, and the BBS1 gene encodes a functional human BBS1 protein;
4) the vector is an AAV2/7m8 vector, the promoter is a rhodopsin kinase (RK) promoter, and the BBS1 gene encodes a functional human BBS1 protein;
5) the vector is an AAV2/7m8 vector, the promoter is a cytomegalovirus immediate-early (CMV) promoter, and the BB S1 gene encodes a functional human BB S1 protein;
6) the vector is an AAV2/7m8 vector, the promoter is a CAG promoter, and the BB S1 gene encodes a functional human BB S1 protein;
7) the vector is an AAV9 vector, the promoter is a rhodopsin kinase (RK) promoter, and the BBS1 gene encodes a functional human BBS1 protein;
8) the vector is an AAV9 vector, the promoter is a cytomegalovirus immediate-early (CMV) promoter, and the BBS1 gene encodes a functional human BBS1 protein; or
9) the vector is an AAV9 vector, the promoter is a CAG promoter, and the BBS1 gene encodes a functional human BB S1 protein.

21.-22. (canceled)

23. A vector according to claim 1, wherein the vector is an AAV2/7m8 vector, the promoter is a cytomegalovirus immediate-early (CMV) promoter, and the BBS1 gene encodes a functional human BBS1 protein.

24. A pharmaceutical composition comprising the vector according to claim 1 and one or more pharmaceutically acceptable excipients.

25. A method of treating retinal degeneration associated with Bardet-Biedl Syndrome (BBS) comprising administering a therapeutically effective amount of a vector according to claim 1 to a patient suffering from BBS, wherein the vector is administered directly to the eye of the patient.

26. The method of claim 25, wherein the vector is administered subretinally or intravitreally.

27. The method of claim 25, wherein the vector is administered by subretinal injection.

28. The method of claim 25, wherein the vector is administered by intravitreal injection.

29.-34. (canceled)
